SEQUENCE LISTING (22 sequences)

SEQ ID NO: 1 - protein, 30 aa, synthetic construct
PDCCRQKTCSCRLYELLHGAGNHAAGILTL

SEQ ID NO: 2 - protein, 28 aa, synthetic construct
CCRQKTCSCRLYELLHGAGNHAAGILTL

SEQ ID NO: 3 - protein, 26 aa, synthetic construct
RQKTCSCRLYELLHGAGNHAAGILTL

SEQ ID NO: 4 - protein, 23 aa, synthetic construct
TCSCRLYELLHGAGNHAAGILTL

SEQ ID NO: 5 - protein, 21 aa, synthetic construct
SCRLYELLHGAGNHAAGILTL

SEQ ID NO: 6 - protein, 19 aa, synthetic construct
RLYELLHGAGNHAAGILTL

SEQ ID NO: 7 - protein, 11 aa, synthetic construct
YGRKKRRQRRR

SEQ ID NO: 8 - protein, 41 aa, synthetic construct
YGRKKRRQRRRPDCCRQKTCSCRLYELLHGAGNHAAGILTL

SEQ ID NO: 9 - protein, 18 aa, synthetic construct
LLIILRRRIRKQAHAHSK

SEQ ID NO: 10 - protein, 10 aa, synthetic construct
RRLSYSRRRF

SEQ ID NO: 11 - protein, 11 aa, synthetic construct
YARKAARQARA

SEQ ID NO: 12 - protein, 27 aa, synthetic construct
GLAFLGFLGAAGSTMGAWSQPKKKRKV

SEQ ID NO: 13 - protein, 20 aa, synthetic construct
KETWWETWWTEWSQPKKRKV

SEQ ID NO: 14 - protein, 22 aa, synthetic construct
MVRRFLVTLRIRRACGPPRVRV

SEQ ID NO: 15 - protein, 30 aa, synthetic construct
MVKSKIGSWILVLFVAMWSDVGLCKKRPKP

SEQ ID NO: 16 - protein, 18 aa, unidentified
KLALKLALKALKAALKLA

SEQ ID NO: 17 - protein, 28 aa, synthetic construct
LSTAADMQGVVTDGMASGLDKDYLKPDD

SEQ ID NO: 18 - protein, 26 aa, synthetic construct
DPKGDPKGVTVTVTVTVTGKGDPKPD

SEQ ID NO: 19 - protein, 6 aa, synthetic construct
PFVYLI

SEQ ID NO: 20 - protein, 22 aa, unidentified
MVTVLFRRLRIRRACGPPRVRV

SEQ ID NO: 21 - protein, 16 aa, synthetic construct
RKKRRRESRKKRRRES

SEQ ID NO: 22 - protein, 22 aa, synthetic construct
KCFQWQRNMRKVRGPPVSCIKR

(19) United States
(12) Patent Application Publication
(10) Pub. No.: US 2025/0376497 A1
(43) Pub. Date: Dec. 11, 2025

(54) ARTIFICIAL PROTEIN AND USES THEREOF

(71) Applicant: ALMA MATER STUDIORUM - UNIVERSITA' DI BOLOGNA, Bologna (BO) (IT)

(72) Inventors: Alessandro SILVANI, Bologna (BO) (IT); Giuseppe PLAZZI, Bologna (BO) (IT); Giorgio MEDICI, Bologna (BO) (IT); Stefania TRAZZI, Bologna (BO) (IT); Elisabetta CIANI, Bologna (BO) (IT); Gabriele MATTEOLI, Bologna (BO) (IT); Sara ALVENTE, Bologna (BO) (IT); Viviana LO MARTIRE, Bologna (BO) (IT); Stefano BASTIANINI, Bologna (BO) (IT); Chiara BERTEOTTI, Bologna (BO) (IT); Giovanna ZOCCOLI, Bologna (BO) (IT)

(73) Assignee: ALMA MATER STUDIORUM - UNIVERSITA' DI BOLOGNA, Bologna (BO) (IT)

(21) Appl. No.: 18/878,422

(22) PCT Filed: Jun. 28, 2023
(86) PCT No.: PCT/EP2023/067633

§ 371 Date: Dec. 23, 2024

(30) Foreign Application Priority Data
Jun. 30, 2022 (IT) 102022000013894

(51) Int. Cl. C07K 14/575; A61K 9/00; A61K 38/00; C07K 14/16; C12N 15/62; C12N 15/85

(52) U.S. Cl. CPC C07K 14/575; A61K 9/0019; A61K 38/00; C07K 14/163; C12N 15/62; C12N 15/85; C07K 2319/10

(57) ABSTRACT

The present invention relates to a synthetic peptide comprising the following elements from the N-terminus to the C-terminus: a) a first cell-penetrating peptide or functional fragments or derivatives, or biologically active variants thereof and b) a second peptide with agonist activity of OR1 and OR2 receptors or functional fragments or derivatives, or biologically active variants thereof.

### FIELD OF THE INVENTION

The present invention relates to a synthetic peptide comprising, from the N-terminus to the C-terminus, a first cell-penetrating peptide or functional fragments or derivatives, or biologically active variants thereof and a second peptide with orexin-receptor-1 (OR1) and orexin-receptor-2 (OR2) agonist activity or functional fragments or derivatives, or biologically active variants thereof. The invention also relates to the therapeutic use of said synthetic peptide.

### PRIOR ART

Orexins A and B (OXA and OXB) (Sakurai et al., 1998), also called hypocretins 1 and 2 (de Lecea et al., 1998), are peptides produced by a population of hypothalamic neurons with maximum activity during active wakefulness and minimum activity during rapid-eye-movement (REM) sleep (Mileykovskiy et al., 2005). Orexins bind two G protein-coupled receptors, designated OR1, which is selective for OXA, and OR2, which is non-selective for OXA and OXB (Sakurai et al., 1998). OR1 and OR2 are widely expressed in the central nervous system (CNS) (Leonard & Kukkonen, 2014), in line with the widespread projections of orexinergic neurons (Peyron et al., 1998). The orexinergic CNS system modulates multiple physiological functions including wake and sleep behavior, energy homeostasis and autonomic control of the cardiovascular system (Bastianini & Silvani, 2018; Grimaldi et al., 2014; Sakurai, 2007). OR1 and OR2 are also expressed outside the CNS, in sites that include adipose tissue, the male reproductive system (Leonard & Kukkonen, 2014), the heart (Perez et al., 2015) and the bone marrow (McAlpine et al., 2019). The orexin receptors outside the CNS may be relevant for the pathophysiology of heart failure (Perez et al., 2015) and atherosclerosis (McAlpine et al., 2019) and may bind orexins that leak into the systemic circulation after their release into the CNS by the hypothalamic neurons that produce them (McAlpine et al., 2019). The OXA peptide is relatively protected from inactivation by peptidases through two disulphide bonds, an N-terminus pyroglutamic residue, and C-terminus amidation (Sakurai et al., 1998). In contrast, OXB is a linear peptide with a free N-terminus (Sakurai et al., 1998) and is rapidly metabolized in the blood (Kastin & Akerstrom, 1999) and even in cerebrospinal fluid (Yoshida et al., 2003). The permeability of the blood-brain barrier (BBB) to OXA is still debated. OXA has been reported to rapidly enter the mouse brain by simple diffusion (Kastin & Akerstrom, 1999), a result in line with other experiments performed on rats (Kodama & Kimura, 2002; Van de Bittner et al., 2018) and with functional data obtained on dogs (John et al., 2000). However, another study reported negligible (<1%) brain penetration of OXA in mice and rats (Bingham et al., 2001), and this conclusion was also supported by data obtained in dogs (Fujiki et al., 2003).

Narcolepsy type 1 (NT1) is a severe and rare (prevalence 14/100,000 subjects (Scheer et al., 2019)) neurological disease associated with an almost complete functional loss of orexinergic neurons (Peyron et al., 2000) probably due to autoimmune damage (Mahoney et al., 2019). NT1 is characterized by a wide range of signs and symptoms including excessive daytime sleepiness, fragmented sleep with increased muscle tone, increased propensity and reduced latency to REM sleep with episodes of REM sleep at the onset of sleep (SOREMs), cataplexy (loss of muscle tone during wakefulness, often evoked by positive emotions), a tendency to obesity, and a non-dipper blood pressure profile (Grimaldi et al., 2014; Mahoney et al., 2019).

A clinical condition similar to human NT1 occurs in dogs due to the mutation of the gene encoding OR2 or as a sporadic form due to orexin deficiency (Mignot, 2014). The clinical picture of NT1 is summarized both in OX-ATX3 transgenic mice with genetic ablation of orexinergic neurons (Hara et al., 2001) and in orexin knockout (OX-KO) mice (Chemelli et al., 1999). Data on double knockout (KO) mice for OR1 and OR2 indicated that the lack of binding of orexins to OR1 and OR2 is necessary for the mouse phenotype to recapitulate the complete clinical picture of human NT1, with OR2 playing the major role (Hasegawa et al., 2014; Willie et al., 2003). In accordance with these data, orexin gene therapy improves the pathological phenotype of OX-ATX3 mice (Mieda et al., 2004) and OX-KO (Liu et al., 2008).

Despite the available knowledge on the physiology of the orexinergic system, none of the currently available therapies for NT1 rely on orexin replacement and all have important limitations in terms of efficacy and side effects (S. W. Black et al., 2017). Pre-clinical data on the efficacy of orexin replacement therapy are mixed. In dogs with the familial form of NT1, the therapeutic efficacy of intravenous OXA has been reported (John et al., 2000) but not confirmed (Fujiki et al., 2003). Intravenous or intrathecal administration of OXA was also ineffective in a single dog with the sporadic form of NT1 (Schatzberg et al., 2004). On the other hand, intracerebroventricular (ICV) (Mieda et al., 2004) or intrathecal (Kaushik et al., 2018) administration of OXA has been shown to be effective in reducing cataplexy in OX-KO mice, where ICV administration of OXA also increased wakefulness (Mieda et al., 2004). Intranasal administration of OXA has been reported to result in brain penetration of OXA comparable to that obtained intravenously in rats (Van de Bittner et al., 2018) and a reduction in REM sleep duration and incidence of SOREMs in a pilot study in NT1 patients (Baier et al., 2011). Overall, these studies indicate that the efficacy of systemically administered OXA is, at best, very limited, and indicate insufficient permeability of the BBB as a limiting factor (S. W. Black et al., 2017).

Significant progress has been made in the development of OR2-selective non-peptide agonists capable of crossing the BBB. Compound 30 (Nagahara et al., 2015), renamed YNT-185 (Irukayama-Tomobe et al., 2017), has been shown to increase wakefulness duration and decrease SOREMs in OX-KO mice after intraperitoneal administration, albeit with limited efficacy. Compound TAK-925 has been shown to increase the time spent awake after subcutaneous (SC) injection in control wild-type (WT) mice (Yukitake et al., 2019) and has passed a phase 1 study in NT1 patients with administration by intravenous infusion (Tanaka et al., 2020). The compound TAK-988 was shown to be orally bioavailable and able to increase the time spent awake and decrease cataplexy in OX-ATX3 mice (Kimura, Ishikawa, & Suzuki, 2020) and increase time spent awake in non-human primates (Kimura, Ishikawa, Hara, et al., 2020). Although all of these selective OR2 receptor agonists have the potential to contribute to NT1 therapy, knowledge of the physiology of the orexinergic system and the pathophysiology of NT1 indicates that the binding of orexins to both OR1 and OR2 receptors would need to be reintegrated to fully resolve the NT1 clinical picture. In particular, it has been shown that the severity of cataplexy is modest in OR2-KO mice, in which the binding of orexins to OR1 is preserved, whereas it is high in OX-KO mice, in which orexins and their binding to both OR1 and OR2 are completely absent (Willie et al., 2003). In double KO mice for OR1 and OR2, OR2 expression in the dorsal raphe via a viral vector was shown to prevent cataplexy, potentially activating the serotonergic neurons of this structure, which physiologically express both OR1 and OR2, but was still unable to prevent the excess of REM sleep during the period of activity (dark) (Hasegawa et al., 2014). Recent data have shown a tendency to atherosclerosis in OX-KO mice, due to the lack of activation of OR1 receptors expressed by hemopoietic precursors in the bone marrow (McAlpine et al., 2019). There is no evidence available on the development of agonists of OR1 and OR2 that are able to cross the BBB after systemic administration and effective in the therapy of NT1.

There is therefore a need to provide new molecules effective in the therapy of NT1.

### DESCRIPTION OF THE INVENTION

The present inventors have now found an agonist of OR1 and OR2, herein defined as OX-DRAGON (TAT-OXA (4-33)), effective on cataplexy, a characteristic sign of NT1 by systemic SC administration.

OX-DRAGON is an artificial peptide consisting of a truncated sequence (4-33) of the native sequence of OXA (said sequence being preferably characterized by the sequence SEQ ID NO: 1), fused at the N-terminus with the sequence of the cell penetrating TAT peptide of type-1 human immunodeficiency virus (HIV) (preferably characterized by the sequence SEQ ID NO: 7). Like the native OXA protein, OX-DRAGON has an amidated residue at the C-terminus and two disulphide bridges (14-20 and 15-22).

OXA is preferably characterized by the sequence identified by NCBI Accession number 1R02_A (protein).

TAT is preferably characterized by a sequence comprised in the sequence identified by NCBI Accession number BAA12992.1 (protein) and D86068.1 (nucleotide sequence).

The structure of OX-DRAGON is adapted to allow it to cross the BBB after systemic administration and to allow it to bind and activate both orexin receptors. In-vitro and in-vivo experiments show that OX-DRAGON crosses cell membranes, acts as an OR1 and OR2 agonist, and exerts a significant anti-cataplectic effect after SC administration in OX-KO mice, a validated mouse model of NT1.

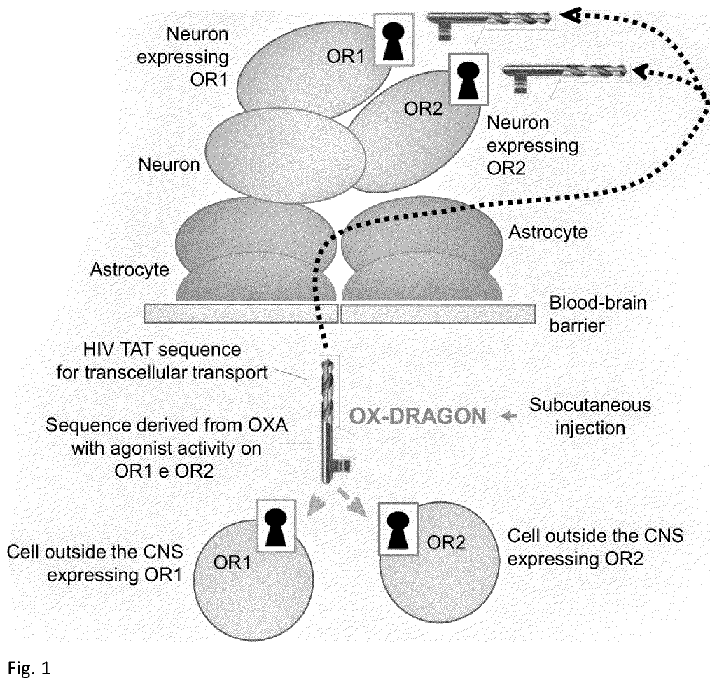

OX-DRAGON is also designed to act on CNS neurons that express orexin receptors as well as on peripheral extracerebral cells that express such receptors, with the potential therefore to completely resolve the clinical picture of NT1 (John et al., 2000; Mieda et al., 2004) (FIG. 1). The TAT peptide crosses the BBB (Bolhassani et al., 2017; Schwarze et al., 1999; Trazzi et al., 2018) and has already been successfully employed for brain delivery of brain-derived neurotrophic factor (BDNF), a peptide whose receptors are expressed on cell membranes, as OR1 and OR2 also are (Verheij et al., 2016; Wu et al., 2015). Based on data available in the literature, OXA may have a limited ability to cross the BBB (John et al., 2000; Kastin & Akerstrom, 1999; Kodama & Kimura, 2002; Van de Bittner et al., 2018). Such ability can synergize with the effect of TAT (Kimura, Ishikawa, Hara, et al., 2020; Kimura, Ishikawa, & Suzuki, 2020; Tanaka et al., 2020; Yukitake et al., 2019) further increasing the ability of OX-DRAGON to cross the BBB. No side effects related to immunogenicity or toxicity of the TAT peptide have been reported so far (Bolhassani et al., 2017). The structure of OXA is fully conserved between humans (Homo sapiens) and mice (Mus musculus) (Sakurai et al., 1998), which supports its preclinical development in the mouse. In addition, OXA is highly resistant to peptidases (Kastin & Akerstrom, 1999; Sakurai et al., 1998; Yoshida et al., 2003). OX-DRAGON includes the C-terminus amidated sequence of OXA, which is critical for binding to OR1 and OR2. The truncated 4-33 sequence of the OXA peptide, which is included in OX-DRAGON, does not have the N-terminus pyroglutamate residue, which is present in the native OXA but which would be technically difficult to bind to the TAT peptide sequence. However, there is evidence that the 4-33 truncated sequence of OXA maintains a substantial agonist potency on OR1 and OR2, with concentrations yielding semi-maximal responses (EC50) equal to approximately 1/7 of those of the native OXA protein, and with the same receptor selectivity profile (EC50 OR1/OR2=1.6) (Lang et al., 2004). In-vitro experiments on cells of neuronal lineage expressing human OR1 or OR2 showed that OX-DRAGON has a similar potency on OR1 and OR2 as OXA, with a receptor selectivity profile more favoring OR2 (EC50 OR1/OR2=6.5). Experiments on mouse models of NT1 also demonstrated that OX-DRAGON is effective after SC administration. The SC route of administration is safe and tolerable, being successfully used by millions of people, including those of paediatric age, for long-term insulin therapy of diabetes.

An object of the present invention is therefore a synthetic peptide comprising the following elements from the N-terminus to the C-terminus:
a) a first cell-penetrating peptide or functional fragments or derivatives, or biologically active variants thereof and
b) a second peptide with agonist activity of OR1 and OR2 or functional fragments or derivatives, or biologically active variants thereof.

Preferably the second peptide comprises or consists of a fragment of the protein orexin A (OXA), preferably wherein said fragment comprises or consists of:
i. the sequence PDCCRQKTCSCRLYELLHGAGNHAAGILTL (SEQ ID NO:1), or
ii. a fragment of SEQ ID No: 1 comprising or consisting of:
   the sequence CCRQKTCSCRLYELLHGAGNHAAGILTL (SEQ ID NO:2) or
   the sequence RQKTCSCRLYELLHGAGNHAAGILTL (SEQ ID NO:3) or
   the sequence TCSCRLYELLHGAGNHAAGILTL (SEQ ID NO:4) or
   the sequence SCRLYELLHGAGNHAAGILTL (SEQ ID NO:5) or
   the sequence RLYELLHGAGNHAAGILTL (SEQ ID NO:6) or
iii. a sequence having a percent identity of at least 70% with a sequence comprising or consisting of SEQ ID NO:1, SEQ ID NO:2, SEQ ID NO:3, SEQ ID NO:4, SEQ ID NO:5 or SEQ ID NO:6,
preferably said second peptide is amidated at the C-terminus,
preferably said second peptide presents disulphide bridges, preferably in positions 3-9 and 4-11 of SEQ ID NO: 1 or in positions 1-7 and 2-9 of SEQ ID NO:2.

Preferably the first peptide comprises or consists of a TAT peptide of the HIV-1 virus, preferably said TAT peptide comprises or consists of a sequence having a percent identity of at least 70% with a sequence comprising or consisting of the sequence YGRKKRRQRRR (SEQ ID NO:7), preferably said first peptide comprises or consists of SEQ ID NO:7, or wherein the first peptide comprises or consists of a sequence having a percent identity of at least 70% with a sequence comprising or consisting of one of the sequences below:

(SEQ ID NO: 9)
LLIILRRRIRKQAHAHSK,
(SEQ ID NO: 10)
RRLSYSRRRF,
(SEQ ID NO: 11)
YARKAARQARA,
(SEQ ID NO: 12)
GLAFLGFLGAAGSTMGAWSQPKKKRKV,
(SEQ ID NO: 13)
KETWWETWWTEWSQPKKRKV,
(SEQ ID NO: 14)
MVRRFLVTLRIRRACGPPRVRV,
(SEQ ID NO: 15)
MVKSKIGSWILVLFVAMWSDVGLCKKRPKP,
(SEQ ID NO: 16)
KLALKLALKALKAALKLA,
(SEQ ID NO: 17)
LSTAADMQGVVTDGMASGLDKDYLKPDD,
(SEQ ID NO: 18)
DPKGDPKGVTVTVTVTVTGKGDPKPD,
(SEQ ID NO: 19)
PFVYLI,
(SEQ ID NO: 20)
MVTVLFRRLRIRRACGPPRVRV,
(SEQ ID NO: 21)
RKKRRRESRKKRRRES
or
(SEQ ID NO: 22)
KCFQWQRNMRKVRGPPVSCIKR.

Preferably, the peptide of the invention comprises or consists of SEQ ID NO:8 (YGRKKRRQRRRPDCCRQKTCSCRLYELLHGAGNHAAGILTL), or functional fragments, equivalents, variants, mutants, derivatives, or functional recombinant or synthetic analogues thereof,
wherein preferably said sequence presents disulphide bridges at positions 14-20 and 15-22 of SEQ ID NO:8 and wherein the C-terminus is amidated.

Preferably the peptide of the invention comprises or consists of SEQ ID NO:8 (YGRKKRRQRRRPDCCRQKTCSCRLYELLHGAGNHAAGILTL),
wherein said sequence presents disulphide bridges at positions 14-20 and 15-22 of SEQ ID NO: 8 and wherein the C-terminus is amidated.

Preferably, the peptide of the invention is anti-cataplectic in action and/or has at least one of the following functions:
promoting wakefulness or contrasting sleepiness
reduction of body weight and glucose intolerance in conditions of obesity
improvement of myocardial function in heart failure
prevention of atherosclerosis
anti-inflammatory action
analgesic action
chemotherapeutic action against colon cancer and neuroblastoma.

A further object of the invention is a pharmaceutical composition comprising the peptide as disclosed herein and at least one pharmaceutically acceptable vehicle, preferably for subcutaneous administration.

Another object of the invention is an isolated nucleic acid encoding the peptide disclosed herein or a recombinant expression vector comprising said isolated nucleic acid.

A further object of the invention is a host cell comprising and/or expressing the peptide as disclosed herein, or the nucleic acid or the vector as disclosed herein.

Further objects of the invention are the peptide or the pharmaceutical composition or the nucleic acid or the vector or the cell as disclosed herein for medical use, preferably for use in the treatment and/or prevention of NT1, narcolepsy type 2, idiopathic hypersomnia, obesity or associated cardio-metabolic comorbidities, including atherosclerosis, of heart failure, of inflammation, for example of septic shock, of neuroinflammation, or of inflammation at the intestinal barrier level, for example in ulcerative colitis, and of tumour and metastasis, for example colon cancer and neuroblastoma; as an analgesic, for example for the management of operative anaesthesia, in the treatment of pain, of drug-resistant pain conditions, such as post-stroke pain, and of pain induced by chemotherapy, of chronic pain, optionally in patients suffering from NT1.

Preferably, the peptide defined herein is for subcutaneous administration.

A further object of the invention is a method for producing the peptide of the invention comprising the steps of transforming a host cell with an expression vector encoding said peptide, culturing said host cell under conditions that allow expression of said fusion protein and optionally recovering and purifying said fusion protein.

The present invention also includes sequences having a percent identity of at least 70% with the sequences disclosed herein, functional fragments or derivatives thereof.

The present invention will be disclosed by means of non-limiting examples, referring to the following figures:

FIG. 1. Rationale for the structure of OX-DRAGON.

OXA: orexin A. OR1 and OR2: orexin receptors 1 and 2. OX-DRAGON: artificial peptide with activity of double agonist of OR1 and OR2 and ability to cross the blood-brain barrier. TAT: transactivator of transcription for human immunodeficiency virus HIV-1 (cell penetrating peptide). CNS: central nervous system. The existence of neurons and non-neuronal cells expressing both OR1 and OR2 has been reported but is omitted from the scheme for the sake of clarity.

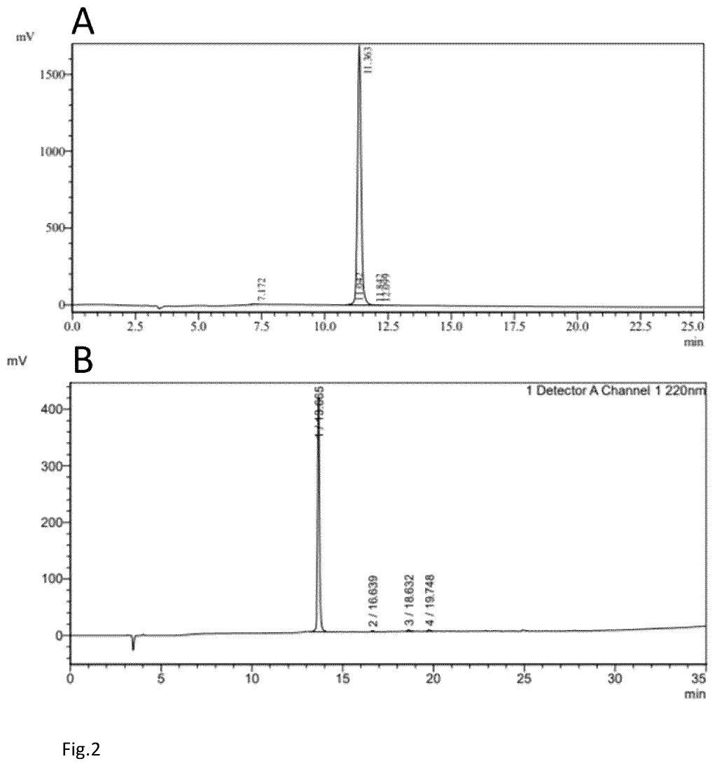

FIG. 2. High pressure liquid chromatography of the two OX-DRAGON batches.

Analyses were performed with solutions of 0.065% trifluoroacetic acid in 100% water (volume/volume) and 0.05% trifluoroacetic acid in 100% acetonitrile (volume/volume), with total flow of 1 mL/min and wavelength of 220 nm. The analysis of the first batch (A) was performed with Alltima™ C18 column 4.6×250 mm; the analysis of the second batch (B) was performed with Inertsil ODS-3 column 4.6×250 mm.

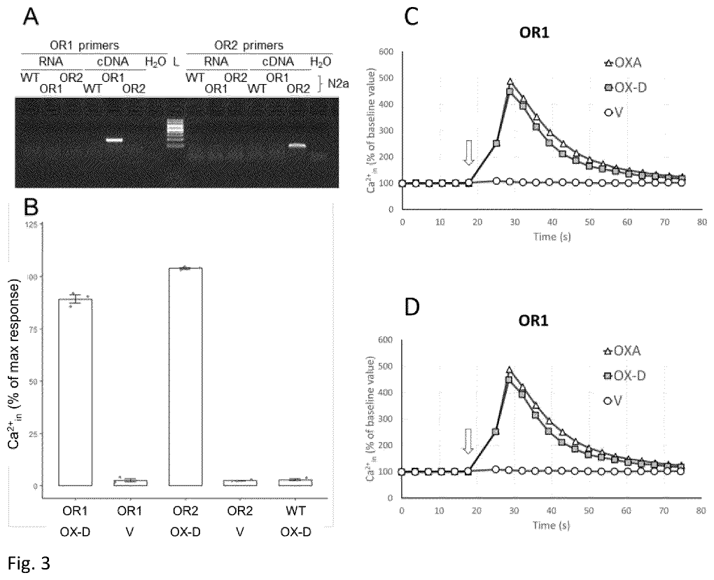

FIG. 3. Effects of OX-DRAGON in vitro.

Panel A: L, ladder DNA. Panel B: Data are reported as mean values and standard errors of the mean on triplicate measurements. The dots indicate the individual measures. Panels C and D show representative results of the time course of intracellular Ca²⁺ concentration (Ca²⁺in) in response to microinjections of OX-DRAGON 8.5 μM or OXA 14.8 μM or saline (vehicle) in wells containing cultured Neuro-2a murine neuroblastoma cells stably expressing human OR1 or OR2. The arrows indicate the time of the microinjection.

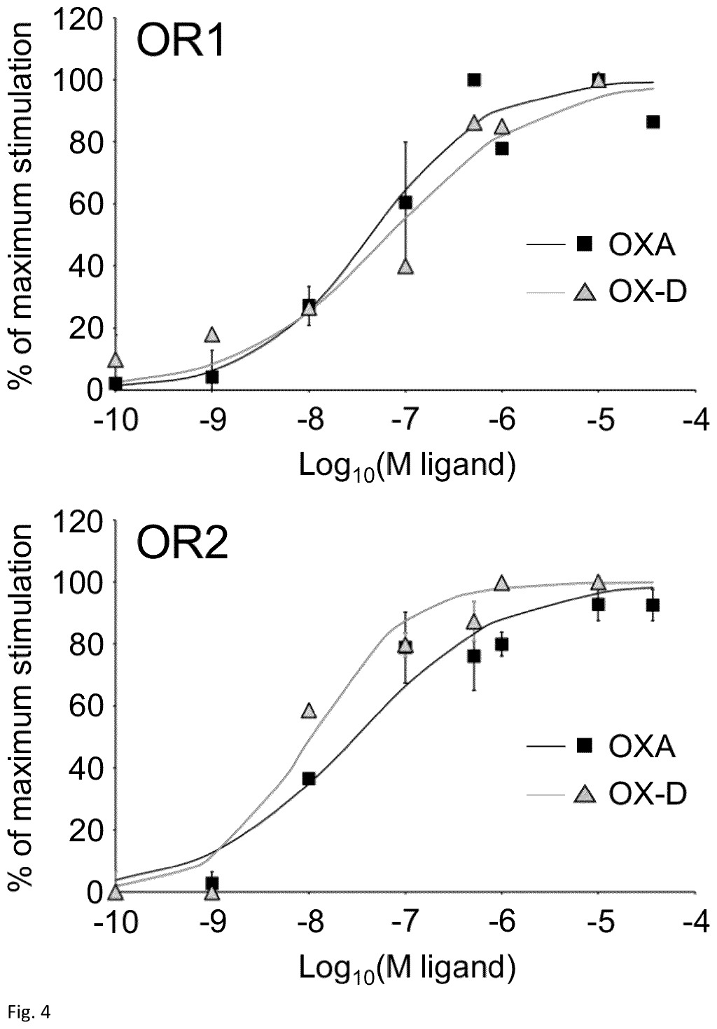

FIG. 4. Concentration-response relationships for OX-DRAGON and OXA on OR1 and OR2. Data are reported as mean values and standard errors of the mean on 1-3 cell batches for different ligands and receptors. Results were obtained on HEK-293T cells using commercial reporter assay kits (n. 600240 and 600250, Cayman Chemical, USA).

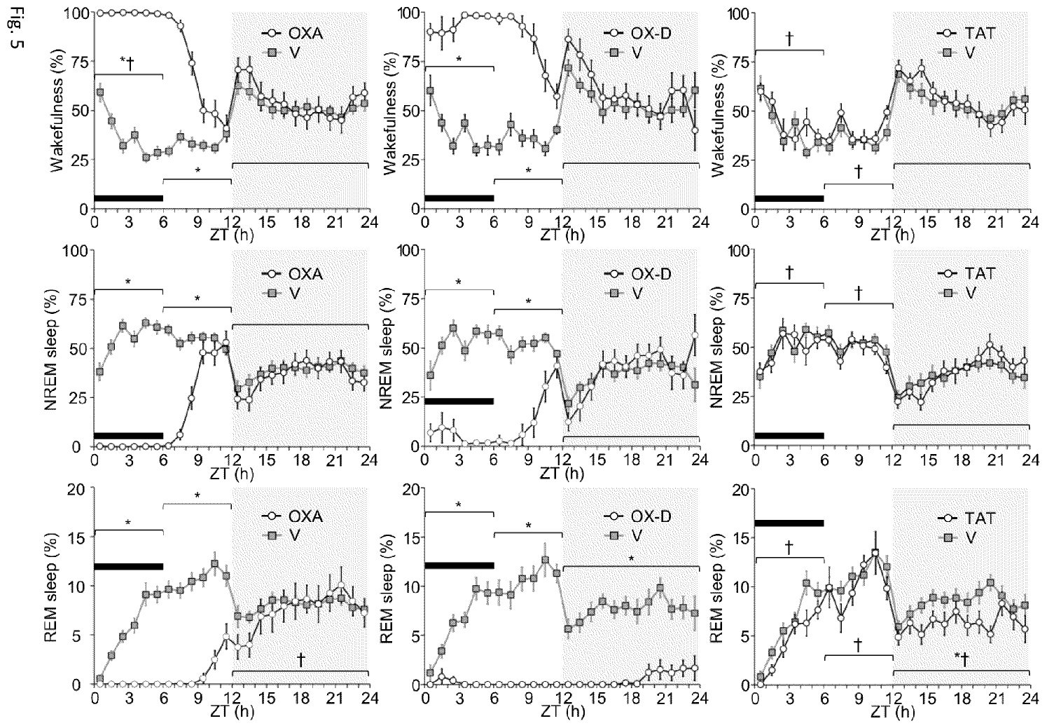

FIG. 5. In-vivo effects of OX-DRAGON, OXA and TAT in OX-KO mice after intracerebroventricular administration.

Wakefulness, non-REM sleep (NREM) and REM sleep are expressed as a percentage of the recording time. V: vehicle (artificial cerebrospinal fluid). OX-D: OX-DRAGON. TAT: TAT peptide. ZT: Zeitgeber time (i.e., time since lights were switched on). Black bars: intracerebroventricular infusion (5 μL/h from ZT0 to ZT6, with vehicle or peptides at 160 μM). * and †: P<0.05, Wilcoxon test with exact significance (Monte Carlo method) vs. vehicle (with correction for false-discovery rate) or vs. OX-DRAGON, respectively. Curves indicate mean values and standard error of the mean with N=20, 8 and 12 for OXA vs. vehicle, OX-DRAGON vs. vehicle and TAT vs. vehicle, respectively.

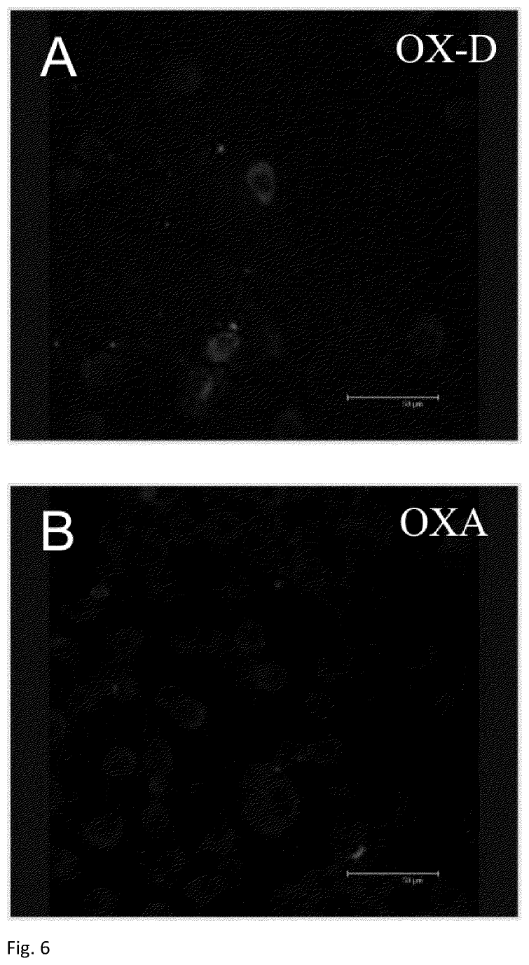

FIG. 6. OX-DRAGON is able to cross cell membranes.

The panels show immunofluorescence imaging (rabbit anti-OXA 16-33 amide primary antibody, 1:200, Phoenix peptides #H-003-36) in Neuro-2a cells without expression of OR1 and OR2. Superposition of the signals in DAPI (nucleic acid) and TRITC (anti-OXA 16-33 amide) fluorescence indicated the presence of TRITC fluorescence signal at the intracellular level in cells exposed to 74 μM OX-DRAGON (OX-D). The presence of diffuse TRITC fluorescence at the cytoplasmic level was confirmed in confocal microscopy (panel A). Results were not replicated in control experiments in cells exposed to 74 μM OXA (panel B). Calibration bars: 50 μm.

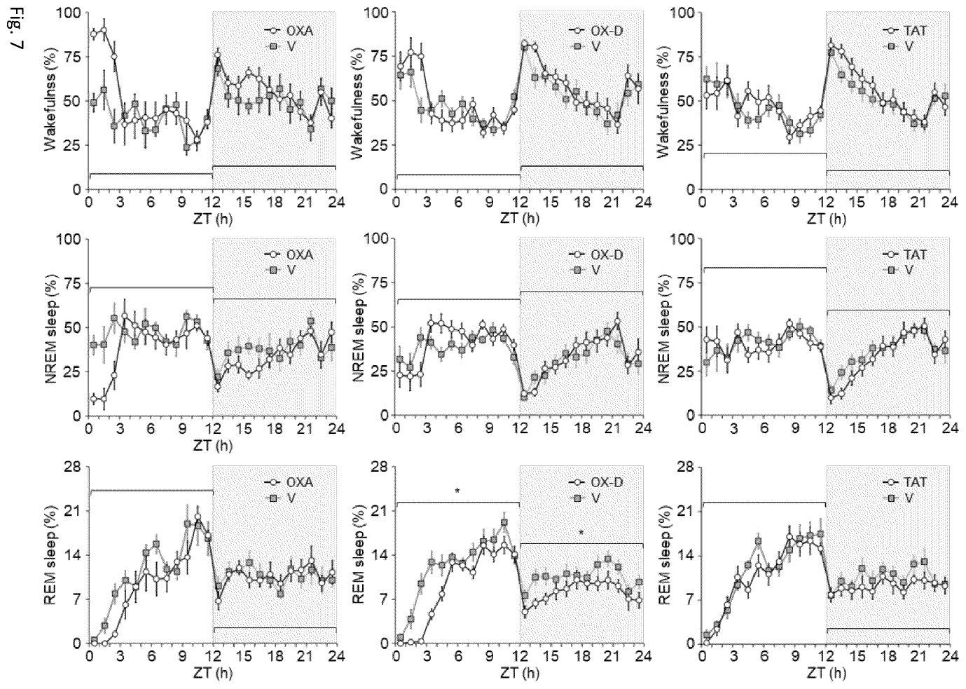

FIG. 7. OX-DRAGON is effective on REM sleep after subcutaneous systemic administration.

Wakefulness, non-REM sleep (NREM) and REM sleep are expressed as a percentage of the recording time. V: vehicle (saline). OX-D: OX-DRAGON. TAT: TAT peptide. ZT: Zeitgeber Time (i.e., time since lights were switched on). Each peptide was injected subcutaneously at the concentration of 160 μL in 1 mL of vehicle at ZT0. *: P<0.05, Wilcoxon test with exact significance (Monte Carlo method) vs. vehicle with correction for false-discovery rate. The curves indicate mean values and standard error of the mean with N=7, 12, and 11 for OXA, OX-DRAGON, and TAT, respectively.

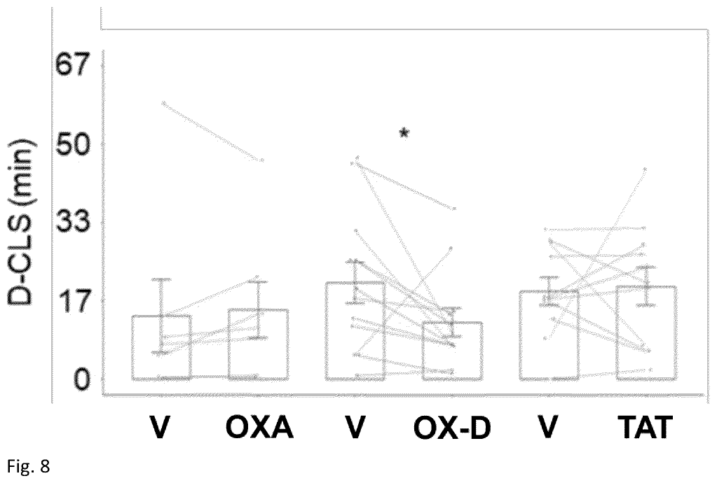

FIG. 8. OX-DRAGON has anti-cataplectic effect after systemic subcutaneous administration.

D-CLS: total duration of cataplectic-like episodes during the dark period. V: vehicle (saline). OX-D: OX-DRAGON. TAT: TAT peptide. Each peptide was injected subcutaneously at the concentration of 160 μL in 1 mL of vehicle at the beginning of the light period. *: P<0.05, two-tailed Wilcoxon test with exact significance (Monte Carlo method) vs. vehicle. The bars indicate mean values and standard error of the mean with N=7, 12, and 11 for OXA, OX-DRAGON, and TAT, respectively. The dots indicate values in individual mice.

### DETAILED DESCRIPTION OF THE INVENTION

In the context of the present invention, synthetic peptide also means an artificial peptide, a fusion peptide, or a conjugate.

In the context of the present invention, although the preferred cell-penetrating peptide is TAT, preferably characterized by the sequence YGRKKRRQRRR (SEQ ID NO:7), any cell-penetrating peptide known to the person skilled in the art can be used, such as for example (Elmquist et al., 2001; Rousselle et al., 2001; Stalmans et al., 2015):

pVEC:
(SEQ ID NO: 9)
LLIILRRRIRKQAHAHSK
SynB3:
(SEQ ID NO: 10)
RRLSYSRRRF.

Other cell-penetrating peptides that could be used are:

| | | |
|---|---|---|
| HIV-1 TATk | YARKAARQARA | SEQ ID NO: 11 |
| MPG | GLAFLGFLGAAGSTMGAWSQPKKKRKV | SEQ ID NO: 12 |
| PEP-1 | KETWWETWWTEWSQPKKRKV | SEQ ID NO: 13 |
| ARF(1-22) | MVRRFLVTLRIRRACGPPRVRV | SEQ ID NO: 14 |
| BPrPr(1-30) | MVKSKIGSWILVLFVAMWSDVGLCKKRPKP | SEQ ID NO: 15 |
| MAP | KLALKLALKALKAALKLA | SEQ ID NO: 16 |
| Azurin p28 | LSTAADMQGVVTDGMASGLDKDYLKPDD | SEQ ID NO: 17 |
| VT5 | DPKGDPKGVTVTVTVTVTGKGDPKPD | SEQ ID NO: 18 |
| C105Y | PFVYLI | SEQ ID NO: 19 |
| M918 | MVTVLFRRLRIRRACGPPRVRV | SEQ ID NO: 20 |
| DPV3 | RKKRRRESRKKRRRES | SEQ ID NO: 21 |
| Human lactoferrin | KCFQWQRNMRKVRGPPVSCIKR | SEQ ID NO: 22 |

In the context of the present invention, the preferred OR1 and OR2 receptor binding sequence is PDCCRQKTCSCRLYELLHGAGNHAAGILTL (SEQ ID NO:1), with C-terminus amidated residue and two disulphide bridges (3-9 and 4-11), which corresponds to the OXA protein truncated sequence 4-33 (Lang et al., 2004).

Other OR1 and OR2 receptor binding sequences that could be used are the reduced sequence SEQ ID NO:1 or fragments thereof, whether or not devoid of disulphide bridges, or the following truncated OXA sequences (Lang et al., 2004), all of which are intended with C-terminus amidated residues, and which constitute portions or fragments, optionally reduced (i.e. devoid of disulphide bridges) of the above preferred sequence (SEQ ID NO: 1):

OXA(6-33)
SEQ ID NO: 2
CCRQKTCSCRLYELLHGAGNHAAGILTL
OXA(8-33)
SEQ ID NO: 3
RQKTCSCRLYELLHGAGNHAAGILTL
OXA(11-33)
SEQ ID NO: 4
TCSCRLYELLHGAGNHAAGILTL
OXA(13-33)
SEQ ID NO: 5
SCRLYELLHGAGNHAAGILTL
OXA(15-33)
SEQ ID NO: 6
RLYELLHGAGNHAAGILTL

The present invention also encompasses modifications concerning sequences intermediate between that of the cell-penetrating peptide and that of the orexinergic agonist, possibly provided with side chains.

Intermediate sequences could for example include peptide tags and/or functional sequences, e.g., endosome escape or protease resistance sequences (Eldridge et al., 2009; Li et al., 2020; Lotze et al., 2016; Varkouhi et al., 2011; Wadia et al., 2004).

The term “functional derivative” is used herein to denote a chemical derivative of the disclosed peptides that has the same physiological function as the corresponding unmodified counterpart or, alternatively, that has the same function in vitro in a functional assay (e.g., in one of the assays disclosed herein or in one of the examples disclosed herein).

A pharmaceutical composition is formulated to be compatible with its intended route of administration. Examples of routes of administration include parenteral, e.g., subcutaneous, intradermal, intravenous, transdermal (topical), transmucosal, and rectal, inhalation, intranasal administration.

The invention also relates to a polynucleotide encoding the peptides as defined herein, a vector comprising the above polynucleotide and a genetically engineered host cell expressing the peptide as defined above. Preferably, the polynucleotide is selected from the group consisting of: RNA or DNA, preferably said polynucleotide is DNA.

Preferably the vector is an expression vector selected from the group consisting of: plasmids, viral particles and phages.

Preferably said host cell is selected from the group consisting of: bacterial cell, fungal cell, insect cell, animal cell and plant cell, preferably said host cell is an animal cell.

The peptides of the invention are in the form of linear and multimeric synthetic or recombinant peptides in any chemical, physical and/or biological form such as to maintain their function. The peptides of the invention can be synthesized and used in the branched form as multiple antigenic peptide (MAP), as disclosed, e.g., in U.S. Pat. No. 5,229,490.

All amino acids in the peptide may have the same stereochemistry, for example the peptide may consist only of L-amino acids or only D-amino acids. Alternatively, the peptide may comprise a combination of L and D amino acids. Also included in the present invention are retroinverse peptides, either partial or total (Rai, 2019).

The peptide of the present invention may be in the form of a dimer or a multimer. In the present description, examples of spacers comprised in the dimer or multimer include ester bonds (—CO—O—, —O—CO—), ether bonds (—O—), amide bonds (NHCO, CONH), linkers based on sugar chains, polyethylene glycol linkers, peptide linkers, and the like. Examples of peptide linkers include linkers containing at least one of the 20 natural amino acids that make up a protein. The number of amino acids of the linker peptide is, for example, but not limited to, 1 to 20, 1 to 15, 1 to 12, 1 to 10, 1 to 8, 1 to 6, or 1 to 4. Examples of peptide linkers include arginine dimer, arginine trimer, arginine tetramer, lysine dimer, lysine trimer, lysine tetramer, glycine dimer, glycine trimer, glycine tetramer, glycine pentamer, glycine hexamer, alanine-alanine-tyrosine-leucine (AAY), isoleucine-leucine-alanine (ILA), arginine-valine-lysine-arginine (RVKR), and the like. The spacer may be bivalent or multivalent.

When the peptide of the present invention is a multimer, a branched multivalent linker (e.g., dendrimer), a metal complex, or the like can be used for the linkage.

Also included in the present invention are derivatives or variants of the peptides defined above or of the invention. Suitably, “derivatives” or “variants” include those in which, instead of the naturally occurring amino acid, the amino acid appearing in the sequence is a structural analogue thereof. The amino acids used in the sequences may also be derivatized or modified, e.g., labelled, provided that the function of the peptide is not significantly adversely affected. Derivatives and variants as disclosed above can be prepared during peptide synthesis or by post-production modification or when the peptide is in recombinant form using the known techniques of site-directed mutagenesis, random mutagenesis, or enzymatic cleavage and/or nucleic acid ligation. In the context of the present invention, variants also include variants with antagonistic activity of OR1 or OR2 or both OR1 and OR2.

The functional “fragments” according to the invention can be obtained by truncation, e.g., by removal of one or more amino acids from the N-terminus end and/or by removal of one or more amino acids within the sequence. Such fragments may be derived from the sequences disclosed herein or may be derived from a functionally equivalent peptide as disclosed above.

Suitably, functional variants or derivatives according to the invention have an amino acid sequence having more than 70%, e.g., 75% or 80%, preferably more than 85%, e.g., more than 90% or 95% homology or identity to the sequences disclosed herein.

The polynucleotides or peptides disclosed here can also be defined in terms of more specific identities and/or similarity ranges to those exemplified here. The sequence identity will typically be above 70%, more preferably above 80%, even more preferably above 90%, and may be above 95%. The identity and/or similarity of a sequence may be 70, 71, 72, 72, 73, 74, 75, 76, 77, 78, 79, 80, 81, 82, 83, 84, 85, 86, 87, 88, 89, 90, 91, 92, 93, 94, 95, 96, 97, 98, or 99% or greater than a sequence exemplified herein. Unless otherwise specified, as used here, the percentage sequence identity and/or similarity of two sequences can be determined using the algorithm from Karlin and Altschul (Karlin & Altschul, 1990), modified as in (Karlin & Altschul, 1993). Such an algorithm is incorporated into the NBLAST and XBLAST programs (Altschul et al., 1990). BLAST searches can be performed with the NBLAST program, score=100, wordlength=12, to obtain sequences with the desired percent sequence identity. To obtain gapped alignments/alignments of sequences with gaps for comparison purposes, Gapped BLAST can be used as disclosed in (Altschul et al., 1997). When using the BLAST and Gapped BLAST programs, the default parameters of the respective programs (NBLAST and XBLAST) can be used. See the NCBI/NIH website.

The peptides of the invention, as defined herein, may be chemically modified, for example post-translationally modified. For example, they may be glycosylated or comprise modified amino acid residues. They may be in a variety of forms of polypeptide derivatives, including starches and conjugates with polypeptides.

Chemically modified peptides also include those having one or more residues chemically derivatized from the reaction of a functional side group. Such derivatized side groups include those which have been derived to form amine hydrochlorides, p-toluene sulfonyl groups, carbobenzoxy groups, t-butyloxycarbonyl groups, chloroacetyl groups and formyl groups. Free carboxylic groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine.

Also included as chemically modified peptides are cyclized peptides, i.e., peptides of the invention that are linked with a covalent bond to generate a ring.

Also included as chemically modified peptides are those that contain one or more naturally occurring amino acid derivatives of the twenty standard amino acids. For example, 4-hydroxyproline can be substituted for proline or homoserine can be substituted for serine.

A peptide of the invention may have a marking label. Suitable labels include radioisotopes, fluorescent labels, enzyme labels or other protein labels such as biotin.

Any formula provided herein is also intended to represent both unlabelled and isotopically labelled forms of the peptides. Isotopically labelled peptides have structures represented by the formulae provided herein except for the fact that one or more atoms are replaced by an atom with a chosen atomic mass or mass number. Examples of isotopes that can be incorporated into the peptides of the invention include isotopes of hydrogen, carbon, nitrogen, oxygen, phosphorus, fluorine, and chlorine. Pharmaceutically acceptable solvates in accordance with the invention include those wherein the solvent of crystallization may be isotopically substituted, e.g., D20, do-acetone, d6-DMSO.

Peptides as disclosed above for use in accordance with the invention may be prepared by conventional synthesis modalities, including genetic or chemical means.

Synthetic techniques, such as solid-phase Merrifield-type synthesis, may be preferred for reasons of purity, antigenic specificity, freedom from unwanted side products, and ease of production. Techniques suitable for solid phase peptide synthesis are well known to those skilled in the art (see for example (Fields & Noble, 1990; Merrifield, 1969)). The chemical synthesis may be performed by methods well known in the art involving cyclic sets of selective deprotection reactions of the functional groups of a terminal amino acid and coupling of selectively protected amino acid residues, followed finally by complete deprotection of all functional groups. The synthesis may be carried out in solution or on a solid support using suitable solid phases known in the art.

In an alternative embodiment, a peptide of the invention can be produced or administered in the form of a polynucleotide that encodes it and is capable of expressing it. Such polynucleotides may be synthesized according to methods that are well known in the art, as disclosed by way of example in (Green et al., 2012). Such polynucleotides may be used in vitro or in vivo in the production of a peptide of the invention. Such polynucleotides can then be administered or used in the treatment of NT1 or of another disease or condition as disclosed herein.

The present invention also includes expression vectors comprising such polynucleotide sequences. Such expression vectors are usually assembled in the art of molecular biology and may for example involve the use of plasmid DNA and suitable primers, promoters, enhancers, and other elements, such as, for example, polyadenylation signals, which may be necessary and which are positioned with the correct orientation, in order to allow the expression of a peptide of the invention. Other suitable vectors are, however, clear to those skilled in the art. As a further example in this regard, we refer to (Green et al., 2012).

Thus, the peptide may be provided by transporting such a vector to a cell and allowing transcription from the vector to occur. Suitably, a polynucleotide of the invention or for use in the invention in a vector may be operatively linked to a control sequence that is capable of providing for expression of the coding sequence by the host cell, i.e., the vector may be an expression vector. The term “operatively linked” refers to a juxtaposition wherein the components disclosed are in a relationship permitting them to function in their intended manner. A regulatory sequence, such as a promoter, “operatively linked” to a coding sequence is positioned in such a way that expression of the coding sequence is obtained under conditions compatible with the regulatory sequence.

The vectors can be, for example, vectors consisting of plasmids, viruses or phages having a replication origin, optionally a promoter for expression of said polynucleotide and optionally a promoter regulator. The vectors may contain one or more selectable marker genes, for example an ampicillin resistance gene in the case of a bacterial plasmid or a resistance gene for a fungal vector. The vectors may be used in vitro, for example for the production of DNA or RNA or used to transfect or transform a host cell, for example a mammalian host cell. The vectors may also be adapted to be used in vivo, e.g., to permit in-vivo expression of the polypeptide.

The invention also includes cells that have been modified to express a peptide of the invention. Such cells include preferably taxonomically higher eukaryotic cell lines, such as mammalian cells or insect cells, lower eukaryotic cells, such as yeast, or prokaryotic cells such as bacterial cells. Particular examples of cells that can be modified by inserting vectors encoding a peptide of the invention include mammalian HEK293T, CHO, HeLa and COS cells. Expression may be obtained in transformed oocytes. A suitable peptide can be expressed in the cells of a transgenic non-human animal, in particular a mouse.

The present invention also extends to antibodies (monoclonal or polyclonal) and antigen-binding fragments thereof (e.g., F (ab) 2, Fab, and Fv fragments, i.e., fragments of the “variable” region of the antibody, comprising the antigen binding site) directed to the peptides as defined above, i.e., which bind to the epitopes present on the peptides and thus bind selectively and specifically to such peptides and which can be used in the methods of the invention.

The peptides of the present invention may be employed alone as the sole therapy or in combination with other therapeutic agents for the prevention and/or treatment of the diseases mentioned above and below.

Also encompassed by the invention are compositions comprising one or more peptides or polynucleotides disclosed herein. Such compositions typically include a pharmaceutically acceptable vehicle. As used herein, the term “pharmaceutically acceptable vehicle” includes, but is not limited to, a saline solution, solvents, dispersion media, coatings, antibacterial and antifungal agents, absorption-delaying and isotonic agents and the like, compatible with pharmaceutical administration. Additional compounds may also be incorporated into the compositions.

A composition may be prepared by methods known in the art of pharmacy. In general, a composition can be formulated to be compatible with its intended route of administration. A formulation may be a solid or a liquid. Administration can be systemic or local. In some aspects, local administration may have advantages for targeted, site-specific management of the disease. Local therapies may provide clinically effective high concentrations directly at the treatment site, with less likelihood of causing systemic side effects.

Examples of routes of administration include parenteral (e.g., intravenous, intradermal, subcutaneous, intraperitoneal, intramuscular) and topical (e.g., epicutaneous, inhalation, transmucosal, and intranasal) administration. Dosage forms suitable for topical administration may include nasal sprays, metered dose inhalers, dry powder inhalers, or nebulization. Solutions or suspensions can include the following components: a sterile diluent such as water for administration, saline solution, non-volatile oils, polyethylene glycols, glycerine, propylene glycol or other synthetic solvents; antibacterial agents such as benzyl alcohol or methyl parabens; antioxidants such as ascorbic acid or sodium bisulphite; chelating agents such as ethylenediaminetetraacetic acid; buffers such as acetates, citrates or phosphates; electrolytes such as sodium ion, chloride ion, potassium ion, calcium ion and magnesium ion and agents for adjusting tonicity such as sodium chloride or dextrose. The pH can be adjusted with acids or bases, such as hydrochloric acid or sodium hydroxide. A composition can be enclosed, for example, in ampoules, disposable syringes or multiple dose vials made of glass or plastic. Compositions may include sterile aqueous solutions or dispersions and sterile powders for the extemporaneous preparation of sterile solutions or dispersions. For intravenous administration, suitable carriers include human albumin, physiological saline, bacteriostatic water, Cremophor EL™ (BASF, Parsippany, N.J.) or phosphate-buffered saline. Typically, a composition is sterile and, when suitable for injectable use, should be fluid so as to allow easy syringeability. It should be stable under the conditions of manufacture and storage and preserved to prevent contamination by microorganisms such as bacteria and fungi. The vehicle can be a solvent or dispersion medium containing, for example, albumin, water, ethanol, a polyol (for example, glycerol, propylene glycol, and liquid polyethylene glycol, and the like), and suitable mixtures thereof. Preventing the action of microorganisms can be achieved by means of various antibacterial and antifungal agents, e.g., parabens, chlorobutanol, phenol, ascorbic acid, thimerosal and the like. In many cases, it will be preferable to include isotonic agents, for example, sugars, polyalcohols such as mannitol, sorbitol, sodium chloride. Prolonged absorption of the injectable compositions can be brought about by including in the composition an agent which delays absorption, for example, aluminium monostearate and gelatin. Sterile solutions can be prepared by incorporating the active compound (e.g., a peptide or polynucleotide disclosed herein) in the required amount in an appropriate solvent with an ingredient or combination of ingredients as enumerated above, as required, followed by sterile filtration. Generally, dispersions are prepared by incorporating the active compound into a sterile vehicle, which contains a dispersion medium and other ingredients such as those listed above. In the case of sterile powders for the preparation of sterile injectable solutions, preparation methods that can be used include vacuum drying and freeze-drying, which produce a powder of the active ingredient and any other desired ingredient from a solution filtered beforehand to make it sterile.

For enteral administration, a composition may be administered, for example, by nasogastric tube, enema, colonoscopy, or orally. Oral compositions may include an inert diluent or an edible vehicle. For the purpose of oral therapeutic administration, the active compound can be incorporated with excipients and used in the form of tablets, troches, or capsules. Oral compositions can also be prepared using a fluid vehicle. Pharmaceutically compatible binding agents can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavouring agent such as peppermint, methyl salicylate, or orange flavouring.

For administration by inhalation, the active compounds may be delivered in the form of an aerosol spray, nebulizer, or inhaler, such as a nasal spray, metered dose inhaler or dry powder inhaler. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be carried out using nasal sprays or nasal suppositories. For transdermal administration, active compounds may be formulated in ointments, salts, gels, or creams as generally known in the art. One example of transdermal administration includes iontophoretic transport to the dermis or other relevant tissues. The active compounds can also be prepared in the form of suppositories (e.g., with conventional suppository bases such as cocoa butter and other glycerides) or retention enemas for rectal delivery. Active compounds can be prepared with carriers that will protect the compound against rapid elimination from the body, such as a controlled release formulation, including implants. Biodegradable, biocompatible polymers can be used, such as ethylene vinyl acetate, polyanhydrides, polyglycolic acid, collagen, polyesters, and polylactic acid. Such formulations can be prepared using standard techniques. The materials can also be obtained commercially. Liposomal suspensions can also be used as pharmaceutically acceptable carriers. These can be prepared according to methods known to those skilled in the art. Transport reagents such as lipids, cationic lipids, phospholipids, liposomes, microencapsulation, and nanoparticles may also be used.

As is common practice, the compositions are normally accompanied by written or printed instructions for use in the treatment in question.

The person skilled in the art will choose the form of administration and the effective dosages, by selecting suitable diluents, adjuvants and/or excipients.

When the peptide of the present invention is in combination with other active ingredients, the active ingredients may be formulated separately in single-ingredient preparations of one of the forms disclosed above and then provided as combined preparations, which are given at the same time or at different times or may be formulated together in a preparation of two or more ingredients.

Peptides as defined above may be administered to a patient in a total daily dose, for example, from 0.1 to 500 mg/kg body weight daily. Dosage unit compositions may contain amounts of submultiples such that they make up the daily dose.

### Example

### 1) the Synthesis of OX-DRAGON is Feasible.

Two batches of OX-DRAGON were synthesized by an external commercial entity (Genscript, (www.genscript.com), with purity ≥98.0% and net peptide content ≥68.0% (FIG. 2). The peptide is highly soluble (<=10 mg/mL) in water, DPBS and DMSO.

### 2) OX-DRAGON is an Agonist of the OR1 and OR2 Receptors for Orexins In Vitro.

OX-DRAGON was shown to evoke maximal in vitro responses in terms of increased cytoplasmic calcium ion concentration ([Ca²⁺]) in two stable clones of mouse neuroblastoma Neuro-2a cells expressing human cDNA for OR1 or OR2, respectively (Holmqvist et al., 2002), without evoking any effect on control Neuro-2a cells that do not express such receptors (wild-type, WT). Selective expression of the OR1- or OR2-coding genes in the corresponding cell lines and the absence of expression of OR1 and OR2 in WT cells were confirmed by real-time PCR from RNA extracted from the individual DNAse-treated and back-transcribed cells (FIG. 3A). Responses in terms of intracellular [Ca²⁺] were assessed in triplicate using Fluo-4 Direct Calcium Assay (Invitrogen) and a Tekan Spark microplate reader, normalizing the results obtained to the maximal responses. The results demonstrated that the responses of OR1-expressing cells (FIGS. 3B and 3C) and particularly those of OR2-expressing cells (FIGS. 3B and 3D) to OX-DRAGON at a concentration of 8.5 μM were close to the maximal ones evoked by OXA at the concentration of 14.8 μM, both in terms of amplitude and in terms of time dynamics. The absence of significant effects of OX-DRAGON on WT cells (FIG. 3B) indicated that the effects of OX-DRAGON on OR1- and OR2-expressing cells are dependent on the respective receptors expressed.

Concentration-response relationships for OX-DRAGON and OXA were estimated at OR1 and OR2 receptors on HEK-293T cells using commercial reporter assay kits (n. 600240 and 600250, Cayman Chemical, USA) (Yamanaka et al., 2020). Data were obtained on 2 cell batches for OXA at OR1, 1 batch for OX-DRAGON at OR1, 3 batches for OXA at OR2, and 2 batches for OX-DRAGON at OR2. Ligand concentrations yielding semimaximal responses (EC50) were estimated fitting data to the Hill equation function (Neubig et al., 2003). The results (FIG. 4) yielded the following EC50 values:
OXA at OR1: 43.7 nM
OXA at OR2: 30.0 nM
OX-DRAGON at OR1: 67.7 nM
OX-DRAGON at OR2: 10.5 nM

This yielded estimates of OR1/OR2 EC50 ratios of 1.45 for OXA and of 6.47 for OX-DRAGON, indicating similar receptor selectivity profiles of OXA and OX-DRAGON, with slightly greater preference of OX-DRAGON for OR2 vs. OR1 with respect to OXA.

3) OX-DRAGON Exerts Similar or Superior Effects to OXA on Wakefulness and Sleep after ICV Administration in the OX-KO Mouse Model of NT1.

To test the ability of OX-DRAGON to exert in-vivo CNS effects similar to those of OXA regardless of its ability to cross the BBB, experiments were performed in a mouse model of NT1 consisting of adult female OX-KO mice (internal breeding Dept Biomedical and Neuromotor Sciences of the University of Bologna, genetic background N>10 C57Bl/6J (Bastianini et al., 2011)), operated under general anaesthesia (isoflurane) for the implantation of electroencephalogram (EEG; bilateral frontoparietal derivation) electrodes of the electromyogram (EMG) of the nuchal muscles (Bastianini et al., 2011), and of a cannula in the lateral cerebral ventricle (C313G/SPC and C313IC/SPC, Plastics One; anteroposterior coordinates −0.6 mm, lateral +1.2 mm, dorsoventral −2.0 mm). A biocompatible acrylic resin protection for was applied to the animal's head to protect the electrode and cannula connectors. After 1-2 weeks of post-operative recovery, the animals were connected via rotating electrical and fluid connectors respectively to thin electrical cables and to a catheter, itself connected to a precision pump for ICV infusion. The rotating connectors, cables and catheter were supported by a balanced arm to allow the animals full freedom of movement during the recordings, which were made with light: dark cycle of 12:12 hours. Wakefulness, non-REM sleep, and REM sleep were assessed based on EEG and EMG tracings with a technique previously published in detail (Bastianini et al., 2011). The administration of OX-DRAGON via ICV (160 μM at 5 μL/h for the first 6 hours of the light period, ZT0-6), to bypass BBB, compared to the corresponding administration of a vehicle (artificial cerebrospinal fluid; FIG. 5, centre), produced consistent effects of increased wakefulness time and decreased non-REM sleep time throughout the light period (ZT0-12). Moreover, a marked reduction in REM sleep time continued until the end of the dark period (ZT24). The effects on wakefulness and non-REM sleep were similar to those evoked by OXA ICV (160 μM at 5 μL/h, ZT0-6; FIG. 5, left), the native double agonist of OR1 and OR2, although with a slightly lower increase in wakefulness time in the ZT1-ZT6 period. In contrast, ICV administration of TAT (160 μM at 5 μL/h, ZT0-6; FIG. 6, right) in OX-KO mice did not evoke significant effects on wakefulness and non-REM sleep time but resulted in a significant reduction in REM sleep time during the dark period (ZT13-24) compared to the vehicle, although with values significantly higher than those obtained with OX-DRAGON (FIG. 5, right). This suggests that the reduction in REM sleep time during the dark period exerted by OX-DRAGON via ICV in OX-KO mice is not solely attributable to the effects of TAT.

### 4) OX-DRAGON is Capable of Crossing Cell Membranes.

Immunofluorescence assays with anti-OXA antibodies (16-33 amide, Phoenix peptides #H-003-36) in confocal microscopy revealed a cytoplasmic signal of diffused fluorescence in WT Neuro-2a cells ((Holmqvist et al., 2002); see point 2 above) treated for 1 hour with OX-DRAGON 74 μM. This demonstrates internalization of OX-DRAGON into cells after 1 hour incubation. This result was not observed in control experiments with OXA 74 μM (FIG. 6). These data demonstrate that the specific chemical structure of OX-DRAGON gives the molecule the ability to cross cell membranes. Because crossing the BBB requires the ability to cross endothelial cell membranes, these experiments support the ability of OX-DRAGON to cross the BBB and reach brain tissue after systemic administration.

5) OX-DRAGON is Effective on REM Sleep after Systemic Subcutaneous Administration in NT1 OX-KO Mouse Model.

Experiments were performed as in step 3 above, except for the absence of the ICV cannula implant. Systemic SC administration of OX-DRAGON (single injection at ZT0, 160 μM in 1 mL saline) in OX-KO mice (internal breeding Dept Biomedical and Neuromotor Sciences of the University of Bologna; (Bastianini et al., 2011)) proved able to significantly decrease the time spent in REM sleep both during the light period and during the dark period, without exerting significant effects on the time spent awake or on the time spent in non-REM sleep (FIG. 7, centre). Equimolar SC injections (160 μM in 1 mL of saline) of OXA (FIG. 7, left) or TAT (FIG. 7, right) did not result in significant effects on the time spent in REM sleep, non-REM sleep or wakefulness. These results demonstrate that OX-DRAGON is effective systemically and suggest that the effect is attributable to the ability of OX-DRAGON to cross the BBB. The results also suggest that the effects of SC administration of OX-DRAGON are not simply achievable by SC administration of OXA, presumably due to the decreased ability of OXA to cross the BBB. Finally, the results indicate that, unlike after ICV administration, the effects of OXA on REM sleep after SC administration are not attributable to non-specific effects of the TAT peptide.

### 6) OX-DRAGON Exerts a Significant Anti-Cataplectic Effect Upon Systemic SC Administration in the NT1 OX-KO Mouse Model.

Systemic administration of OX-DRAGON (single injection at ZT0, 160 μM in 1 mL saline) in OX-KO mice (internal breeding Dept Biomedical and Neuromotor Sciences of the University of Bologna; (Bastianini et al., 2011); experiments disclosed in point 5 above) proved able to significantly decrease the total duration of cataplexy-like episodes during the dark period (FIG. 8). Such episodes, identified as direct transitions from wakefulness to REM sleep (Scammell et al., 2009), during the dark period are 100% specific for cataplexy in mouse models of NT1 (Fujiki et al., 2009). Correspondence between such events on polysomnographic tracings and abrupt behavioural transitions to a state of immobility was confirmed based on synchronized video tracings in a subset of mice (n=4) during treatment with vehicle and with OX-DRAGON. The effect of OX-DRAGON was due to a reduction in the number of cataplexy-like episodes (P=0.034, Wilcoxon one-tailed test with exact Monte Carlo significance), with no significant changes in their duration. This effect was not replicated by equimolar SC injections of either OXA or TAT (FIG. 8). These results indicate that OX-DRAGON has the potential to become a novel therapeutic agent for NT1 with anti-cataplectic action.

### Application

NT1 has a relatively high estimated prevalence (14/100000 (Scheer et al., 2019)) compared to other rare diseases and carries significant human, social and economic costs (J. Black et al., 2014). None of the currently available therapies for NT1 are based on the replacement of orexins of which patients are deficient, and all have important limitations in terms of efficacy and side effects (S. W. Black et al., 2017). OX-DRAGON may become a new therapy for NT1 strictly based on the aetiology and pathophysiology of the disease. In particular, OX-DRAGON is an agonist of both types of orexin receptors and may, therefore, allow complete compensation for the lack of orexin in NT1 patients. On this basis, OX-DRAGON can have a substantial medical, social, and economic impact on NT1 patients and their families.

Because of the pleiotropic physiological effects of orexins, OX-DRAGON has surprisingly diverse possible therapeutic applications, which include, in addition to NT1, type 2 narcolepsy, obesity, heart failure, emergence from anaesthesia, pain and inflammation, and cancer.

In particular, TAK925, a OR2 agonist, has recently also been shown to be effective in promoting wakefulness in patients with type 2 narcolepsy, which is characterized by lack of cataplexy and by detectable orexin levels in cerebrospinal fluid (Tanaka et al., 2020). In the same way, even in light of the experiments already carried out by the present authors, OX-DRAGON could have therapeutic utility even in patients suffering from type 2 narcolepsy. This would further increase the social impact of OX-DRAGON, considering that the prevalence of narcolepsy without cataplexy (type 2) is almost four times higher than that of NT1 (Scheer et al., 2019), and might extend also to patients with idiopathic hypersomnia. Obesity is a severe health problem worldwide (Swinburn et al., 2011). The orexin system affects the control of energy balance and metabolism. Consequently, NT1 increases the risk of obesity, particularly in childhood (Grimaldi et al., 2014; Poli et al., 2013). Enhancement of the OR2-mediated signalling pathway has been shown to prevent diet-induced obesity and improve leptin sensitivity and glucose tolerance in mice (Funato et al., 2009). On the other hand, recent data also indicate a distinct role of OR1 in energy metabolism, showing that orexin binding to OR1 is sufficient to prevent diet-induced obesity in OR2 KO mice fed a high-fat diet (Kakizaki et al., 2019). OX-DRAGON, which is a dual agonist of OR1 and OR2, may, therefore, also be useful for the treatment of obesity and associated cardiometabolic comorbidities, not only in patients and, particularly, in children with NT1, but also in the general population.

There is evidence that the human heart expresses OR2 receptors and that these are relevant to the pathophysiology of heart failure (Perez et al., 2015). In particular, OR2 expression at the cardiac level was significantly higher in human hearts with dilated or ischemic cardiomyopathy than in control hearts. This could represent a mechanism of compensation: mice KO for OR2 show signs of cardiac ventricular diastolic dysfunction, while the infusion of OXA, a non-selective agonist of OR1 and OR2, reduces cardiac dysfunction in a mouse model of heart failure (Perez et al., 2015). These results suggest that OX-DRAGON, which is designed to be an OR1 and OR2 agonist, such as OXA, and is able to permeate the cell membrane, which can increase its transcapillary transport, can also be used for the therapy of heart failure.

There is strong preclinical evidence that OXA facilitates emergence from anaesthesia with propofol (Zhang et al., 2012) and isoflurane (Yang et al., 2019) with mechanisms that include OR1-mediated modulation of GABA-A receptors (Sachidanandan et al., 2017) and dorsal raphe serotoninergic neurons (Yang et al., 2019). OR1 agonists have the potential to provide anaesthetists with tools to actively promote emergence from anaesthesia after surgery (Zhou et al., 2018) and OX-DRAGON may prove useful in this regard as well. These considerations could also be relevant for patients with NT1, as the paucity of available evidence does not yet allow us to conclude whether or not such patients present an increased perioperative risk (Hershner et al., 2019).

The first results that identified analgesic properties of OXA (Bingham et al., 2001) were subsequently confirmed and extended, highlighting a role of OR1 at the spinal level (Yamamoto et al., 2002) and of the periaqueduct grey matter of the midbrain (Ho et al., 2011). Persistent pain and stress activate orexinergic pathways that inhibit pain, suggesting the involvement of orexins as endogenous pain modulators (Watanabe et al., 2005). Preclinical evidence suggests that OR1 agonists may also be effective against drug-resistant pain conditions, such as post-stroke pain (Matsuura et al., 2020) and chemotherapy-induced pain (Toyama et al., 2017). OX-DRAGON can therefore also be useful in the analgesic field. Again, this may also be relevant for patients with NT1, in whom a high prevalence of chronic pain has been reported (Cremaschi et al., 2019; Dauvilliers et al., 2011).

OR1 agonists such as OX-DRAGON can also be useful for treating severe inflammatory conditions. Systemic administration of OXA in a mouse model of advanced septic shock, when the BBB is compromised, acts on the CNS to modulate inflammation and increase survival (Ogawa et al., 2016). Early administration of OX-DRAGON, which might cross the intact BBB, might therefore be helpful in acting early and preventing full septic shock. In addition, OXA can exert anti-inflammatory effects by also binding to peripheral OR1 receptors. Recent data indicate that OXA can act on OR1 at the gut level to prevent lipopolysaccharide-induced neuroinflammation at the gut barrier level (Tunis et al., 2019) and to decrease inflammation in ulcerative colitis (Messal et al., 2018). In addition, OXA acts via OR1 on pre-neutrophils of the bone marrow to attenuate myelopoiesis, curbing the nocturnal increase of circulating inflammatory monocytes and neutrophils and limiting the severity and extent of atherosclerosis (McAlpine et al., 2019).

There is evidence that activation of OR1 results in a pro-apoptotic effect in colon cancer and neuroblastoma cell lines (Rouet-Benzineb et al., 2004). These results were subsequently confirmed on human colon cancer cell lines and on liver metastases, both in vitro and in vivo, after xenograft in nude mice (Voisin et al., 2011). OXA also promotes robust apoptosis in cells resistant to 5-fluorouracil, the most commonly used chemotherapy in colon cancer, and reverses the development of tumours when administered seven days after their inoculation in the mouse model (Rouet-Benzineb et al., 2004). OXA could promote tumour apoptosis in vivo by directly activating caspase-3. These results suggest that OX-DRAGON, which has agonist activity on OR1 and is capable of permeating the cell membrane, which can increase its transcapillary transport, can be used as a therapy for colon cancer (Rouet-Benzineb et al., 2004) and neuroblastoma. Recent data on cell lines of human colon cancer indicate that OXA induces autophagy (Wen et al., 2016) and that activation of OR1 and cholecystokinin A receptors that form receptor heterodimers with them reduces cell migration (Bai et al., 2017). Orexin-dependent apoptosis could be mediated by motifs present in both OR1 and OR2, by the involvement of phosphotyrosine phosphatase SHP2 and by the induction of mitochondrial apoptosis (Mogavero et al., 2021).

### REFERENCES

Altschul, S. F., Gish, W., Miller, W., Myers, E. W., & Lipman, D. J. (1990). Basic local alignment search tool. Journal of Molecular Biology, 215 (3), 403-410. https://doi.org/10.1016/S0022-2836 (05) 80360-2
Altschul, S. F., Madden, T. L., Schäffer, A. A., Zhang, J., Zhang, Z., Miller, W., & Lipman, D. J. (1997). Gapped BLAST and PSI-BLAST: A new generation of protein database search programs. Nucleic Acids Research, 25 (17), 3389-3402. https://doi.org/10.1093/nar/25.17.3389
Bai, B., Chen, X., Zhang, R., Wang, X., Jiang, Y., Li, D., Wang, Z., & Chen, J. (2017). Dual-agonist occupancy of orexin receptor 1 and cholecystokinin A receptor heterodimers decreases G-protein-dependent signaling and migration in the human colon cancer cell line HT-29. Biochimica et Biophysica Acta. Molecular Cell Research, 1864 (7), 1153-1164. https://doi.org/10.1016/j.bbamcr.2017.03.003
Baier, P. C., Hallschmid, M., Seeck-Hirschner, M., Weinhold, S. L., Burkert, S., Diessner, N., Göder, R., Aldenhoff, J. B., & Hinze-Selch, D. (2011). Effects of intranasal hypocretin-1 (orexin A) on sleep in narcolepsy with cataplexy. Sleep Medicine, 12 (10), 941-946. https://doi.org/10.1016/j.sleep.2011.06.015
Bastianini, S., & Silvani, A. (2018). Clinical implications of basic research: The role of hypocretin/orexin neurons in the central autonomic network. Clinical and Translational Neuroscience, 2 (2), 2514183X1878932. https://doi.org/10.1177/2514183X18789327
Bastianini, S., Silvani, A., Berteotti, C., Elghozi, J.-L., Franzini, C., Lenzi, P., Lo Martire, V., & Zoccoli, G. (2011). Sleep related changes in blood pressure in hypocretin-deficient narcoleptic mice. Sleep, 34 (2), 213-218. https://doi.org/10.1093/sleep/34.2.213
Bingham, S., Davey, P. T., Babbs, A. J., Irving, E. A., Sammons, M. J., Wyles, M., Jeffrey, P., Cutler, L., Riba, I., Johns, A., Porter, R. A., Upton, N., Hunter, A. J., & Parsons, A. A. (2001). Orexin-A, an hypothalamic peptide with analgesic properties. Pain, 92 (1-2), 81-90. https://doi.org/10.1016/s0304-3959 (00) 00470-x
Black, J., Reaven, N. L., Funk, S. E., McGaughey, K., Ohayon, M., Guilleminault, C., Ruoff, C., & Mignot, E. (2014). The Burden of Narcolepsy Disease (BOND) study: Health-care utilization and cost findings. Sleep Medicine, 15 (5), 522-529. https://doi.org/10.1016/j.sleep.2014.02.001
Black, S. W., Yamanaka, A., & Kilduff, T. S. (2017). Challenges in the development of therapeutics for narcolepsy. Progress in Neurobiology, 152, 89-113. https://doi.org/10.1016/j.pneurobio.2015.12.002
Bolhassani, A., Jafarzade, B. S., & Mardani, G. (2017). In vitro and in vivo delivery of therapeutic proteins using cell penetrating peptides. Peptides, 87, 50-63. https://doi.org/10.1016/j.peptides.2016.11.011
Chemelli, R. M., Willie, J. T., Sinton, C. M., Elmquist, J. K., Scammell, T., Lee, C., Richardson, J. A., Williams, S. C., Xiong, Y., Kisanuki, Y., Fitch, T. E., Nakazato, M., Hammer, R. E., Saper, C. B., & Yanagisawa, M. (1999). Narcolepsy in orexin knockout mice: Molecular genetics of sleep regulation. Cell, 98 (4), 437-451. https://doi.org/10.1016/s0092-8674 (00) 81973-x
Cremaschi, R. C., Hirotsu, C., Tufik, S., & Coelho, F. M. (2019). Chronic pain in narcolepsy type 1 and type 2—An underestimated reality. Journal of Sleep Research, 28 (3), e12715. https://doi.org/10.1111/jsr.12715
Dauvilliers, Y., Bayard, S., Shneerson, J. M., Plazzi, G., Myers, A. J., & Garcia-Borreguero, D. (2011). High pain frequency in narcolepsy with cataplexy. Sleep Medicine, 12 (6), 572-577. https://doi.org/10.1016/j.sleep.2011.01.010
de Lecea, L., Kilduff, T. S., Peyron, C., Gao, X., Foye, P. E., Danielson, P. E., Fukuhara, C., Battenberg, E. L., Gautvik, V. T., Bartlett, F. S., Frankel, W. N., van den Pol, A. N., Bloom, F. E., Gautvik, K. M., & Sutcliffe, J. G. (1998). The hypocretins: Hypothalamus-specific peptides with neuroexcitatory activity. Proceedings of the National Academy of Sciences of the United States of America, 95 (1), 322-327. https://doi.org/10.1073/pnas.95.1.322
Eldridge, B., Cooley, R. N., Odegrip, R., McGregor, D. P., Fitzgerald, K. J., & Ullman, C. G. (2009). An in vitro selection strategy for conferring protease resistance to ligand binding peptides. Protein Engineering, Design & Selection: PEDS, 22 (11), 691-698. https://doi.org/10.1093/protein/gzp052
Elmquist, A., Lindgren, M., Bartfai, T., & Langel, Ü. (2001). VE-Cadherin-Derived Cell-Penetrating Peptide, pVEC, with Carrier Functions. Experimental Cell Research, 269 (2), 237-244. https://doi.org/10.1006/excr.2001.5316
Fields, G. B., & Noble, R. L. (1990). Solid phase peptide synthesis utilizing 9-fluorenylmethoxycarbonyl amino acids. International Journal of Peptide and Protein Research, 35 (3), 161-214. https://doi.org/10.1111/j.1399-3011.1990.tb00939.x
Fujiki, N., Cheng, T., Yoshino, F., & Nishino, S. (2009). Specificity of direct transition from wake to REM sleep in orexin/ataxin-3 transgenic narcoleptic mice. Experimental Neurology, 217 (1), 46-54. https://doi.org/10.1016/j.expneurol.2009.01.015
Fujiki, N., Yoshida, Y., Ripley, B., Mignot, E., & Nishino, S. (2003). Effects of IV and ICV hypocretin-1 (orexin A) in hypocretin receptor-2 gene mutated narcoleptic dogs and IV hypocretin-1 replacement therapy in a hypocretin-ligand-deficient narcoleptic dog. Sleep, 26 (8), 953-959. https://doi.org/10.1093/sleep/26.8.953
Funato, H., Tsai, A. L., Willie, J. T., Kisanuki, Y., Williams, S. C., Sakurai, T., & Yanagisawa, M. (2009). Enhanced orexin receptor-2 signaling prevents diet-induced obesity and improves leptin sensitivity. Cell Metabolism, 9 (1), 64-76. https://doi.org/10.1016/j.cmet.2008.10.010
Green, M. R., Sambrook, J., & Sambrook, J. (2012). Molecular cloning: A laboratory manual (4th ed.). Cold Spring Harbor Laboratory Press.
Grimaldi, D., Silvani, A., Benarroch, E. E., & Cortelli, P. (2014). Orexin/hypocretin system and autonomic control: New insights and clinical correlations. Neurology, 82 (3), 271-278. https://doi.org/10.1212/WNL.0000000000000045
Hara, J., Beuckmann, C. T., Nambu, T., Willie, J. T., Chemelli, R. M., Sinton, C. M., Sugiyama, F., Yagami, K., Goto, K., Yanagisawa, M., & Sakurai, T. (2001). Genetic ablation of orexin neurons in mice results in narcolepsy, hypophagia, and obesity. Neuron, 30 (2), 345-354. https://doi.org/10.1016/s0896-6273 (01) 00293-8
Hasegawa, E., Yanagisawa, M., Sakurai, T., & Mieda, M. (2014). Orexin neurons suppress narcolepsy via 2 distinct efferent pathways. The Journal of Clinical Investigation, 124 (2), 604-616. https://doi.org/10.1172/JCI71017
Hershner, S., Dauvilliers, Y., Chung, F., Singh, M., Wong, J., Gali, B., Kakkar, R., Mignot, E., Thorpy, M., & Auckley, D. (2019). Knowledge Gaps in the Perioperative Management of Adults With Narcolepsy: A Call for Further Research. Anesthesia and Analgesia, 129 (1), 204-211. https://doi.org/10.1213/ANE.0000000000004088
Ho, Y.-C., Lee, H.-J., Tung, L.-W., Liao, Y.-Y., Fu, S.-Y., Teng, S.-F., Liao, H.-T., Mackie, K., & Chiou, L.-C. (2011). Activation of orexin 1 receptors in the periaqueductal gray of male rats leads to antinociception via retrograde endocannabinoid (2-arachidonoylglycerol)-induced disinhibition. The Journal of Neuroscience, 31 (41), 14600-14610. https://doi.org/10.1523/JNEUROSCI.2671-11.2011
Holmqvist, T., Akerman, K. E. O., & Kukkonen, J. P. (2002). Orexin signaling in recombinant neuron-like cells. FEBS Letters, 526 (1-3), 11-14. https://doi.org/10.1016/s0014-5793 (02) 03101-0
Irukayama-Tomobe, Y., Ogawa, Y., Tominaga, H., Ishikawa, Y., Hosokawa, N., Ambai, S., Kawabe, Y., Uchida, S., Nakajima, R., Saitoh, T., Kanda, T., Vogt, K., Sakurai, T., Nagase, H., & Yanagisawa, M. (2017). Nonpeptide orexin type-2 receptor agonist ameliorates narcolepsy-cataplexy symptoms in mouse models. Proceedings of the National Academy of Sciences of the United States of America, 114 (22), 5731-5736. https://doi.org/10.1073/pnas. 1700499114
John, J., Wu, M. F., & Siegel, J. M. (2000). Systemic administration of hypocretin-1 reduces cataplexy and normalizes sleep and waking durations in narcoleptic dogs. Sleep Research Online, 3 (1), 23-28.
Kakizaki, M., Tsuneoka, Y., Takase, K., Kim, S. J., Choi, J., Ikkyu, A., Abe, M., Sakimura, K., Yanagisawa, M., & Funato, H. (2019). Differential Roles of Each Orexin Receptor Signaling in Obesity. IScience, 20, 1-13. https://doi.org/10.1016/j.isci.2019.09.003
Karlin, S., & Altschul, S. F. (1990). Methods for assessing the statistical significance of molecular sequence features by using general scoring schemes. Proceedings of the National Academy of Sciences of the United States of America, 87 (6), 2264-2268. https://doi.org/10.1073/pnas.87.6.2264
Karlin, S., & Altschul, S. F. (1993). Applications and statistics for multiple high-scoring segments in molecular sequences. Proceedings of the National Academy of Sciences of the United States of America, 90 (12), 5873-5877. https://doi.org/10.1073/pnas.90.12.5873
Kastin, A. J., & Akerstrom, V. (1999). Orexin A but not orexin B rapidly enters brain from blood by simple diffusion. The Journal of Pharmacology and Experimental Therapeutics, 289 (1), 219-223.
Kaushik, M. K., Aritake, K., Imanishi, A., Kanbayashi, T., Ichikawa, T., Shimizu, T., Urade, Y., & Yanagisawa, M. (2018). Continuous intrathecal orexin delivery inhibits cataplexy in a murine model of narcolepsy. Proceedings of the National Academy of Sciences of the United States of America, 115 (23), 6046-6051. https://doi.org/10.1073/pnas.1722686115
Kimura, H., Ishikawa, T., Hara, H., & Suzuki, M. (2020). A novel, orally available orexin 2 receptor-selective agonist with fast off-rate, TAK-994, induces wake-promoting effects in non-human primates. Journal of Sleep Research, 29 (S1), 175-176.
Kimura, H., Ishikawa, T., & Suzuki, M. (2020). TAK-988, an orally available orexin 2 receptor-selective agonist, ameliorates narcolepsy-like symptoms in an orexin neuron-ablated mouse model of narcolepsy. Journal of Sleep Research, 29 (S1), 175.
Kodama, T., & Kimura, M. (2002). Arousal effects of orexin-A correlate with GLU release from the locus coeruleus in rats. Peptides, 23 (9), 1673-1681. https://doi.org/10.1016/s0196-9781 (02) 00109-2
Lang, M., Söll, R. M., Dürrenberger, F., Dautzenberg, F. M., & Beck-Sickinger, A. G. (2004). Structure-activity studies of orexin a and orexin B at the human orexin 1 and orexin 2 receptors led to orexin 2 receptor selective and orexin 1 receptor preferring ligands. Journal of Medicinal Chemistry, 47 (5), 1153-1160. https://doi.org/10.1021/jm030982t
Leonard, C. S., & Kukkonen, J. P. (2014). Orexin/hypocretin receptor signalling: A functional perspective. British Journal of Pharmacology, 171 (2), 294-313. https://doi.org/10.1111/bph.12296
Li, C., Cao, X.-W., Zhao, J., & Wang, F.-J. (2020). Effective Therapeutic Drug Delivery by GALA3, an Endosomal Escape Peptide with Reduced Hydrophobicity. The Journal of Membrane Biology, 253 (2), 139-152. https://doi.org/10.1007/s00232-020-00109-2
Liu, M., Thankachan, S., Kaur, S., Begum, S., Blanco-Centurion, C., Sakurai, T., Yanagisawa, M., Neve, R., & Shiromani, P. J. (2008). Orexin (hypocretin) gene transfer diminishes narcoleptic sleep behavior in mice. The European Journal of Neuroscience, 28 (7), 1382-1393. https://doi.org/10.1111/j.1460-9568.2008.06446.x
Lotze, J., Reinhardt, U., Seitz, O., & Beck-Sickinger, A. G. (2016). Peptide-tags for site-specific protein labelling in vitro and in vivo. Molecular BioSystems, 12 (6), 1731-1745. https://doi.org/10.1039/c6mb00023a
Mahoney, C. E., Cogswell, A., Koralnik, I. J., & Scammell, T. E. (2019). The neurobiological basis of narcolepsy. Nature Reviews. Neuroscience, 20 (2), 83-93. https://doi.org/10.1038/s41583-018-0097-x
Matsuura, W., Nakamoto, K., & Tokuyama, S. (2020). Involvement of descending pain control system regulated by orexin receptor signaling in the induction of central post-stroke pain in mice. European Journal of Pharmacology, 874, 173029. https://doi.org/10.1016/j.ejphar.2020.173029
McAlpine, C. S., Kiss, M. G., Rattik, S., He, S., Vassalli, A., Valet, C., Anzai, A., Chan, C. T., Mindur, J. E., Kahles, F., Poller, W. C., Frodermann, V., Fenn, A. M., Gregory, A. F., Halle, L., Iwamoto, Y., Hoyer, F. F., Binder, C. J., Libby, P., . . . . Swirski, F. K. (2019). Sleep modulates haematopoiesis and protects against atherosclerosis. Nature, 566 (7744), 383-387. https://doi.org/10.1038/s41586-019-0948-2
Merrifield, R. B. (1969). Solid-phase peptide synthesis. Advances in Enzymology and Related Areas of Molecular Biology, 32, 221-296. https://doi.org/10.1002/9780470122778.ch6
Messal, N., Fernandez, N., Dayot, S., Gratio, V., Nicole, P., Prochasson, C., Chantret, I., LeGuilloux, G., Jarry, A., Couvelard, A., Tréton, X., Voisin, T., Ogier-Denis, E., & Couvineau, A. (2018). Ectopic expression of OXIR in ulcerative colitis mediates anti-inflammatory effect of orexin-A. Biochimica et Biophysica Acta. Molecular Basis of Disease, 1864 (11), 3618-3628. https://doi.org/10.1016/j.bbadis.2018.08.023
Mieda, M., Willie, J. T., Hara, J., Sinton, C. M., Sakurai, T., & Yanagisawa, M. (2004). Orexin peptides prevent cataplexy and improve wakefulness in an orexin neuron-ablated model of narcolepsy in mice. Proceedings of the National Academy of Sciences of the United States of America, 101 (13), 4649-4654. https://doi.org/10.1073/pnas.0400590101
Mignot, E. J. M. (2014). History of narcolepsy at Stanford University. Immunologic Research, 58 (2-3), 315-339. https://doi.org/10.1007/s12026-014-8513-4
Mileykovskiy, B. Y., Kiyashchenko, L. I., & Siegel, J. M. (2005). Behavioral correlates of activity in identified hypocretin/orexin neurons. Neuron, 46 (5), 787-798. https://doi.org/10.1016/j.neuron.2005.04.035
Mogavero, M. P., DelRosso, L. M., Fanfulla, F., Bruni, O., & Ferri, R. (2021). Sleep disorders and cancer: State of the art and future perspectives. Sleep Medicine Reviews, 56, 101409. https://doi.org/10.1016/j.smrv.2020.101409
Nagahara, T., Saitoh, T., Kutsumura, N., Irukayama-Tomobe, Y., Ogawa, Y., Kuroda, D., Gouda, H., Kumagai, H., Fujii, H., Yanagisawa, M., & Nagase, H. (2015). Design and Synthesis of Non-Peptide, Selective Orexin Receptor 2 Agonists. Journal of Medicinal Chemistry, 58 (20), 7931-7937. https://doi.org/10.1021/acs.jmedchem.5b00988
Neubig, R. R., Spedding, M., Kenakin, T., Christopoulos, A.; International Union of Pharmacology Committee on Receptor Nomenclature and Drug Classification. (2003). International Union of Pharmacology Committee on Receptor Nomenclature and Drug Classification. XXXVIII. Update on terms and symbols in quantitative pharmacology. Pharmacology Reviews, 55:597-606. https://doi.org/10.1124/pr.55.4.4.
Ogawa, Y., Irukayama-Tomobe, Y., Murakoshi, N., Kiyama, M., Ishikawa, Y., Hosokawa, N., Tominaga, H., Uchida, S., Kimura, S., Kanuka, M., Morita, M., Hamada, M., Takahashi, S., Hayashi, Y., & Yanagisawa, M. (2016). Peripherally administered orexin improves survival of mice with endotoxin shock. ELife, 5, e21055. https://doi.org/10.7554/eLife.21055
Perez, M. V., Pavlovic, A., Shang, C., Wheeler, M. T., Miller, C. L., Liu, J., Dewey, F. E., Pan, S., Thanaporn, P. K., Absher, D., Brandimarto, J., Salisbury, H., Chan, K., Mukherjee, R., Konadhode, R. P., Myers, R. M., Sedehi, D., Scammell, T. E., Quertermous, T., . . . Ashley, E. A. (2015). Systems Genomics Identifies a Key Role for Hypocretin/Orexin Receptor-2 in Human Heart Failure. Journal of the American College of Cardiology, 66 (22), 2522-2533. https://doi.org/10.1016/j.jacc.2015.09.061
Peyron, C., Faraco, J., Rogers, W., Ripley, B., Overeem, S., Charnay, Y., Nevsimalova, S., Aldrich, M., Reynolds, D., Albin, R., Li, R., Hungs, M., Pedrazzoli, M., Padigaru, M., Kucherlapati, M., Fan, J., Maki, R., Lammers, G. J., Bouras, C., . . . . Mignot, E. (2000). A mutation in a case of early onset narcolepsy and a generalized absence of hypocretin peptides in human narcoleptic brains. Nature Medicine, 6 (9), 991-997. https://doi.org/10.1038/79690
Peyron, C., Tighe, D. K., van den Pol, A. N., de Lecea, L., Heller, H. C., Sutcliffe, J. G., & Kilduff, T. S. (1998). Neurons containing hypocretin (orexin) project to multiple neuronal systems. The Journal of Neuroscience, 18 (23), 9996-10015.
Poli, F., Pizza, F., Mignot, E., Ferri, R., Pagotto, U., Taheri, S., Finotti, E., Bernardi, F., Pirazzoli, P., Cicognani, A., Balsamo, A., Nobili, L., Bruni, O., & Plazzi, G. (2013). High prevalence of precocious puberty and obesity in childhood narcolepsy with cataplexy. Sleep, 36 (2), 175-181. https://doi.org/10.5665/sleep.2366
Rai, J. (2019). Peptide and protein mimetics by retro and retroinverso analogs. Chemical Biology & Drug Design, 93 (5), 724-736. https://doi.org/10.1111/cbdd.13472
Rouet-Benzineb, P., Rouyer-Fessard, C., Jarry, A., Avondo, V., Pouzet, C., Yanagisawa, M., Laboisse, C., Laburthe, M., & Voisin, T. (2004). Orexins acting at native OX (1) receptor in colon cancer and neuroblastoma cells or at recombinant OX (1) receptor suppress cell growth by inducing apoptosis. The Journal of Biological Chemistry, 279 (44), 45875-45886. https://doi.org/10.1074/jbc.M404136200
Rousselle, C., Smirnova, M., Clair, P., Lefauconnier, J. M., Chavanieu, A., Calas, B., Scherrmann, J. M., & Temsamani, J. (2001). Enhanced delivery of doxorubicin into the brain via a peptide-vector-mediated strategy: Saturation kinetics and specificity. The Journal of Pharmacology and Experimental Therapeutics, 296 (1), 124-131.
Sachidanandan, D., Reddy, H. P., Mani, A., Hyde, G. J., & Bera, A. K. (2017). The Neuropeptide Orexin-A Inhibits the GABAA Receptor by PKC and Ca2+/CaMKII-Dependent Phosphorylation of Its β1 Subunit. Journal of Molecular Neuroscience: MN, 61 (4), 459-467. https://doi.org/10.1007/s12031-017-0886-0
Sakurai, T. (2007). The neural circuit of orexin (hypocretin): Maintaining sleep and wakefulness. Nature Reviews. Neuroscience, 8 (3), 171-181. https://doi.org/10.1038/nrn2092
Sakurai, T., Amemiya, A., Ishii, M., Matsuzaki, I., Chemelli, R. M., Tanaka, H., Williams, S. C., Richardson, J. A., Kozlowski, G. P., Wilson, S., Arch, J. R., Buckingham, R. E., Haynes, A. C., Carr, S. A., Annan, R. S., McNulty, D. E., Liu, W. S., Terrett, J. A., Elshourbagy, N. A., . . . . Yanagisawa, M. (1998). Orexins and orexin receptors: A family of hypothalamic neuropeptides and G protein-coupled receptors that regulate feeding behavior. Cell, 92 (4), 573-585. https://doi.org/10.1016/s0092-8674 (00) 80949-6
Scammell, T. E., Willie, J. T., Guilleminault, C., Siegel, J. M., & International Working Group on Rodent Models of Narcolepsy. (2009). A consensus definition of cataplexy in mouse models of narcolepsy. Sleep, 32 (1), 111-116.
Schatzberg, S. J., Cutter-Schatzberg, K., Nydam, D., Barrett, J., Penn, R., Flanders, J., deLahunta, A., Lin, L., & Mignot, E. (2004). The effect of hypocretin replacement therapy in a 3-year-old Weimaraner with narcolepsy. Journal of Veterinary Internal Medicine, 18 (4), 586-588. https://doi.org/10.1892/0891-6640 (2004) 18<586: teohrt>2.0.co;2
Scheer, D., Schwartz, S. W., Parr, M., Zgibor, J., Sanchez-Anguiano, A., & Rajaram, L. (2019). Prevalence and incidence of narcolepsy in a US health care claims database, 2008-2010. Sleep, 42 (7), zsz091. https://doi.org/10.1093/sleep/zsz091
Schwarze, S. R., Ho, A., Vocero-Akbani, A., & Dowdy, S. F. (1999). In vivo protein transduction: Delivery of a biologically active protein into the mouse. Science (New York, N.Y.), 285 (5433), 1569-1572. https://doi.org/10.1126/science.285.5433.1569
Stalmans, S., Bracke, N., Wynendaele, E., Gevaert, B., Peremans, K., Burvenich, C., Polis, I., & De Spiegeleer, B. (2015). Cell-Penetrating Peptides Selectively Cross the Blood-Brain Barrier In Vivo. PLOS ONE, 10 (10), e0139652. https://doi.org/10.1371/journal.pone.0139652
Swinburn, B. A., Sacks, G., Hall, K. D., McPherson, K., Finegood, D. T., Moodie, M. L., & Gortmaker, S. L. (2011). The global obesity pandemic: Shaped by global drivers and local environments. Lancet, 378 (9793), 804-814. https://doi.org/10.1016/S0140-6736 (11) 60813-1
Tanaka, S. I., Evans, R., Alexander, R., Imazaki, M., Touno, S., Shimizu, K., Wu, J., Faessel, H., Ratti, E., & Hartman, D. (2020). Selective orexin 2 receptor agonist TAK-925 to treat narcolepsy: Results of a randomized, double-blind, placebo-controlled, multiple-ascending-dose, phase 1 study in patients with narcolepsy type 1. Journal of Sleep Research, 29 (S1), 72.
Toyama, S., Shimoyama, N., & Shimoyama, M. (2017). The analgesic effect of orexin-A in a murine model of chemotherapy-induced neuropathic pain. Neuropeptides, 61, 95-100. https://doi.org/10.1016/j.npep.2016.12.007
Trazzi, S., De Franceschi, M., Fuchs, C., Bastianini, S., Viggiano, R., Lupori, L., Mazziotti, R., Medici, G., Lo Martire, V., Ren, E., Rimondini, R., Zoccoli, G., Bartesaghi, R., Pizzorusso, T., & Ciani, E. (2018). CDKL5 protein substitution therapy rescues neurological phenotypes of a mouse model of CDKL5 disorder. Human Molecular Genetics, 27 (9), 1572-1592. https://doi.org/10.1093/hmg/ddy064
Tunisi, L., Forte, N., Fernández-Rilo, A. C., Mavaro, I., Capasso, R., D'Angelo, L., Milić, N., Cristino, L., Di Marzo, V., & Palomba, L. (2019). Orexin-A Prevents Lipopolysaccharide-Induced Neuroinflammation at the Level of the Intestinal Barrier. Frontiers in Endocrinology, 10, 219. https://doi.org/10.3389/fendo.2019.00219
Van de Bittner, G. C., Van de Bittner, K. C., Wey, H.-Y., Rowe, W., Dharanipragada, R., 10 Ying, X., Hurst, W., Giovanni, A., Alving, K., Gupta, A., Hoekman, J., & Hooker, J. M. (2018). Positron Emission Tomography Assessment of the Intranasal Delivery Route for Orexin A. ACS Chemical Neuroscience, 9 (2), 358-368. https://doi.org/10.1021/acschemneuro.7b00357
Varkouhi, A. K., Scholte, M., Storm, G., & Haisma, H. J. (2011). Endosomal escape pathways for delivery of biologicals. Journal of Controlled Release: Official Journal of the Controlled Release Society, 151 (3), 220-228. https://doi.org/10.1016/j.jconrel.2010.11.004
Verheij, M. M. M., Vendruscolo, L. F., Caffino, L., Giannotti, G., Cazorla, M., Fumagalli, F., Riva, M. A., Homberg, J. R., Koob, G. F., & Contet, C. (2016). Systemic Delivery of a Brain-Penetrant TrkB Antagonist Reduces Cocaine Self-Administration and Normalizes TrkB Signaling in the Nucleus Accumbens and Prefrontal Cortex. The Journal of Neuroscience, 36 (31), 8149-8159. https://doi.org/10.1523/JNEUROSCI.2711-14.2016
Voisin, T., El Firar, A., Fasseu, M., Rouyer-Fessard, C., Descatoire, V., Walker, F., Paradis, V., Bedossa, P., Henin, D., Lehy, T., & Laburthe, M. (2011). Aberrant expression of OX1 receptors for orexins in colon cancers and liver metastases: An openable gate to apoptosis. Cancer Research, 71 (9), 3341-3351. https://doi.org/10.1158/0008-5472.CAN-10-3473
Wadia, J. S., Stan, R. V., & Dowdy, S. F. (2004). Transducible TAT-HA fusogenic peptide enhances escape of TAT-fusion proteins after lipid raft macropinocytosis. Nature Medicine, 10 (3), 310-315. https://doi.org/10.1038/nm996
Watanabe, S., Kuwaki, T., Yanagisawa, M., Fukuda, Y., & Shimoyama, M. (2005). Persistent pain and stress activate pain-inhibitory orexin pathways. Neuroreport, 16 (1), 5-8. https://doi.org/10.1097/00001756-200501190-00002
Wen, J., Zhao, Y., & Guo, L. (2016). Orexin A induces autophagy in HCT-116 human colon cancer cells through the ERK signaling pathway. International Journal of Molecular Medicine, 37 (1), 126-132. https://doi.org/10.3892/ijmm.2015.2409
Willie, J. T., Chemelli, R. M., Sinton, C. M., Tokita, S., Williams, S. C., Kisanuki, Y. Y., Marcus, J. N., Lee, C., Elmquist, J. K., Kohlmeier, K. A., Leonard, C. S., Richardson, J. A., Hammer, R. E., & Yanagisawa, M. (2003). Distinct narcolepsy syndromes in Orexin receptor-2 and Orexin null mice: Molecular genetic dissection of Non-REM and REM sleep regulatory processes. Neuron, 38 (5), 715-730. https://doi.org/10.1016/s0896-6273 (03) 00330-1
Wu, Y., Luo, X., Liu, X., Liu, D., Wang, X., Guo, Z., Zhu, L., Tian, Q., Yang, X., & Wang, J.-Z. (2015). Intraperitoneal Administration of a Novel TAT-BDNF Peptide Ameliorates Cognitive Impairments via Modulating Multiple Pathways in Two Alzheimer's Rodent Models. Scientific Reports, 5, 15032. https://doi.org/10.1038/srep15032
Yamamoto, T., Nozaki-Taguchi, N., & Chiba, T. (2002). Analgesic effect of intrathecally administered orexin-A in the rat formalin test and in the rat hot plate test. British Journal of Pharmacology, 137 (2), 170-176. https://doi.org/10.1038/sj.bjp.0704851
Yamanaka, M., Iwata, H., Masuda, K., Araki, M., Okuno, Y., Okamura, M., Koiwa, J., Tanaka, T. (2020). A novel orexin antagonist from a natural plant was discovered using zebrafish behavioural analysis. European Review for Medical and Pharmacological Sciences, 24:5127-5139. https://doi.org/10.26355/eurrev_202005_21207
Yang, C., Zhang, L., Hao, H., Ran, M., Li, J., & Dong, H. (2019). Serotonergic neurons in the dorsal raphe nucleus mediate the arousal-promoting effect of orexin during isoflurane anesthesia in male rats. Neuropeptides, 75, 25-33. https://doi.org/10.1016/j.npep.2019.03.004
Yoshida, Y., Fujiki, N., Maki, R. A., Schwarz, D., & Nishino, S. (2003). Differential kinetics of hypocretins in the cerebrospinal fluid after intracerebroventricular administration in rats. Neuroscience Letters, 346 (3), 182-186. https://doi.org/10.1016/s0304-3940 (03) 00571-8
Yukitake, H., Fujimoto, T., Ishikawa, T., Suzuki, A., Shimizu, Y., Rikimaru, K., Ito, M., Suzuki, M., & Kimura, H. (2019). TAK-925, an orexin 2 receptor-selective agonist, shows robust wake-promoting effects in mice. Pharmacology, Biochemistry, and Behavior, 187, 172794. https://doi.org/10.1016/j.pbb.2019.172794
Zhang, L.-N., Li, Z.-J., Tong, L., Guo, C., Niu, J.-Y., Hou, W.-G., & Dong, H.-L. (2012). Orexin-A facilitates emergence from propofol anesthesia in the rat. Anesthesia and Analgesia, 115 (4), 789-796. https://doi.org/10.1213/ANE.0b013e3182645ea3
Zhou, W., Cheung, K., Kyu, S., Wang, L., Guan, Z., Kurien, P. A., Bickler, P. E., & Jan, L. Y. (2018). Activation of orexin system facilitates anesthesia emergence and pain control. Proceedings of the National Academy of Sciences of the United States of America, 115 (45), E10740-E10747. https://doi.org/10.1073/pnas.1808622115

1. A synthetic peptide comprising from the N terminus to the C terminus the elements:
a) a first cell-penetrating peptide or functional fragments or derivatives, or biologically active variants thereof and
b) a second peptide with agonist activity of OR1 and OR2 receptors or functional fragments or derivatives, or biologically active variants thereof.

2. The peptide according to claim 1, wherein the second peptide comprises a fragment of the orexin A (OXA) protein.

3. The peptide according to claim 1, wherein the first peptide comprises a human immunodeficiency virus type I (HIV-1) transactivator of transcription (TAT) peptide.

4. The synthetic peptide according to claim 1 comprising SEQ ID NO: 8 (YGRKKRRQRRRPDCCRQKTCSCRLYELLHGAGNHAAGILTL), or its functional fragments, equivalents, variants, mutants, derivatives or functional recombinant or synthetic analogues.

5. The synthetic peptide according to claim 1 comprising or consisting of SEQ ID NO: 8 (YGRKKRRQRRRPDCCRQKTCSCRLYELLHGAGNHAAGILTL),
wherein said sequence presents disulfide bridges in positions 14-20 and 15-22 of SEQ ID NO: 8 and in which the C-terminus is amidated.

6. The peptide according to claim 1 with anti-cataplectic action and/or having at least one of the following functions:
promoting wakefulness or contrasting sleepiness
reducing body weight and glucose intolerance in conditions of obesity
improvement of myocardial function in heart failure
prevention of atherosclerosis
anti-inflammatory action
analgesic action or
chemotherapeutic action against colon cancer and neuroblastoma.

7. A pharmaceutical composition comprising the peptide according to claim 1 and at least one pharmaceutically acceptable vehicle, optionally for subcutaneous administration.

8. An isolated nucleic acid coding for the peptide according to claim 1 or a recombinant expression vector comprising said isolated nucleic acid.

9. A host cell comprising and/or expressing the peptide according to any claim 1.

10. (canceled)

11. A method for the treatment and/or prevention of a condition selected from the group consisting of type 1 narcolepsy, narcolepsy type 2, idiopathic hypersomnia, obesity or associated cardio-metabolic comorbidities including atherosclerosis, of heart failure, of inflammation, of neuroinflammation, or of inflammation at the intestinal barrier level and of tumor and metastases operative anesthesia, the treatment of pain, drug-resistant pain conditions, pain induced by chemotherapy, and chronic pain, optionally in patients with type 1 narcolepsy (NT1), comprising administering a peptide of claim 1 to a patient in need thereof.

12. (canceled)

13. The peptide according to claim 2, wherein the fragment of the orexin A (OXA) protein comprises:
i. the sequence PDCCRQKTCSCRLYELLHGAGNHAAGILTL (SEQ ID NO:1), or
ii. a fragment of SEQ ID NO:1 which comprises:
the sequence CCRQKTCSCRLYELLHGAGNHAAGILTL (SEQ ID NO:2) or
the sequence RQKTCSCRLYELLHGAGNHAAGILTL (SEQ ID NO:3) or
the sequence TCSCRLYELLHGAGNHAAGILTL (SEQ ID NO:4) or
the sequence SCRLYELLHGAGNHAAGILTL (SEQ ID NO:5) or
the sequence RLYELLHGAGNHAAGILTL (SEQ ID NO:6) or
iii. a sequence having a percent identity of at least 70% with a sequence comprising SEQ ID NO: 1, SEQ ID NO:2, SEQ ID NO:3, SEQ ID NO:4, SEQ ID NO:5 or SEQ ID NO:6.

14. The peptide according to claim 1, wherein said second peptide is amidated at the C-terminus, or presents disulfide bridges.

15. The peptide according to claim 14, wherein disulfide bridges are in positions 3-9 and 4-11 of SEQ ID NO:1 or in positions 1-7 and 2-9 of SEQ ID NO: 2.

16. The peptide according to claim 3, wherein said TAT peptide comprises a sequence having a percent identity of at least 70% with a sequence comprising the sequence YGRKKRRQRRR (SEQ ID NO: 7).

17. The peptide according to claim 3, wherein said first peptide comprises SEQ ID NO: 7.

18. The peptide according to claim 3, wherein the first peptide comprises a sequence having a percent identity of at least 70% with a sequence comprising one of the following sequences:
(SEQ ID NO: 9)
LLIILRRRIRKQAHAHSK,
(SEQ ID NO: 10)
RRLSYSRRRF,
(SEQ ID NO: 11)
YARKAARQARA,
(SEQ ID NO: 12)
GLAFLGFLGAAGSTMGAWSQPKKKRKV,
(SEQ ID NO: 13)
KETWWETWWTEWSQPKKRKV,
(SEQ ID NO: 14)
MVRRFLVTLRIRRACGPPRVRV,
(SEQ ID NO: 15)
MVKSKIGSWILVLFVAMWSDVGLCKKRPKP,
(SEQ ID NO: 16)
KLALKLALKALKAALKLA,
(SEQ ID NO: 17)
LSTAADMQGVVTDGMASGLDKDYLKPDD,
(SEQ ID NO: 18)
DPKGDPKGVTVTVTVTVTGKGDPKPD,
(SEQ ID NO: 19)
PFVYLI,
(SEQ ID NO: 20)
MVTVLFRRLRIRRACGPPRVRV,
(SEQ ID NO: 21)
RKKRRRESRKKRRRES
or
(SEQ ID NO: 22)
KCFQWQRNMRKVRGPPVSCIKR.

19. The peptide according to claim 4, wherein said sequence presents disulfide bridges in positions 14-20 and 15-22 of SEQ ID NO: 8 and in which the C-terminus is amidated.
